(19) United States
(12) Patent Application Publication
(10) Pub. No.: US 2005/0069947 A1
(43) Pub. Date: Mar. 31, 2005

(54) Antibodies specific for nanotubes and related methods and compositions

(76) Inventors: Bernard F. Erlanger, Whitestone, NY (US); Louis Brue, Hastings, NY (US); Michael P. Sheetz, Leonia, NJ (US)

Correspondence Address: Cooper & Dunham, 1185 Avenue of the Americas, New York, NY, 10036, US

(21) Appl. No.: 10/484,031

(22) PCT Filed: Jul. 16, 2002
(86) PCT No.: PCT/US02/22620

Related U.S. Application Data
(60) Provisional application No. 60/305,929, filed on Jul. 16, 2001.
(60) Provisional application No. 60/371,023, filed on Apr. 8, 2002.

(51) Int. Cl. G01N 33/53; C07K 16/00

(52) U.S. Cl. 435/7.1; 530/387.1

(57) ABSTRACT

This invention provides two compositions. The first composition comprises a nanotube and at least one anti-nanotube antibody, wherein the anti-nanotube antibody is bound to the nanotube. The second composition comprises a fullerene and at least one anti-fullerene antibody, wherein the anti-fullerene antibody is bound to the fullerene. Finally, this invention provides methods and kits relating to the antibody and compositions of matter.

This invention is a continuation-in-part and claims the benefit of U.S. Provisional Application No. 60/305,929, filed Jul. 16, 2001, and U.S. Provisional Application No. 60/371,023, filed Apr. 8, 2002, the contents of which are hereby incorporated by reference into this application.

This invention was made with support under Grant No. HL 47377-03 from the NIH and under Contract FG02-98ER 14861 from the DOE Basic Energy Sciences. Accordingly, the United States Government has certain rights in the invention.

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

### BACKGROUND OF THE INVENTION

The recent interest in using Buckminster fullerene (fullerene) derivatives in biological systems raises the possibility of their assay by immunological procedures. This, in turn, leads to the question of the ability of these unprecedented polygonal structures, made up solely of carbon atoms, to induce the Production of specific antibodies. Immunization of mice with a C₆₀ fullerene derivative conjugated to bovine thyroglobulin yielded a population of fullerene-specific antibodies of the IgG isotype, showing that the immune repertoire was diverse enough to recognize and process fullerenes as protein conjugates. The population of antibodies included a subpopulation that crossreacted with a C₇₀ fullerene as determined by immune precipitation and ELISA procedures. These assays were made possible by the synthesis of water-soluble fullerene derivatives, including bovine and rabbit serum albumin conjugates and derivatives of trilysine and pentalysine, all of which were characterized as to the extent of substitution and their UV-Vis spectra. Possible interactions of fullerenes with the combining sites of IgG are discussed based on the physical chemistry of fullerenes and previously described protein-fullerene interactions. They remain to be confirmed by the isolation of monoclonal antibodies (mAbs) for X-ray crystallographic studies.

Until 1985 there were only two known allotropic forms of carbon: graphite and diamond. In 1985, a novel allotrope was reported in which 60 carbon atoms were arranged as a truncated icosahedron, with 60 vertices and 32 faces, 12 of which were pentagonal and 20 hexagonal (1). It was dubbed Buckminsterfullerene (usually shortened to fullerene) because of its geodesic character, a name that has held through the present day. A detailed background of metallofullerenes is provided in “Fourth Series of Experiments”, section I (A) (infra).

Considerable activity followed this discovery particularly after procedures were developed to prepare fullerenes in workable quantities (2, 3). Various fullerene-based compounds have been prepared, and diverse uses were sought for them. Some were incorporated into photovoltaic cells (4) and nanotubes (5). Others were tested for biological activity (6), including antiviral (7, 8), antioxidant (9, 10), and chemotactic activities (11), and as neuroprotective agents in a mouse model of amyotrophic lateral sclerosis (12).

Practical application of fullerenes as biological or pharmacological agents requires that dosage and serum levels be capable of measurement, preferably by sensitive, simple immunological procedures. This, in turn, requires that specific antibodies to fullerenes be produced.

The clonal selection theory tells us that antigens elicit the production of antibodies by selecting for specific antibody producing cells already present in the repertoire of immunized animals (13). Although there is debate about the size of the “available” repertoire (14, 15), immunologists usually work on the assumption that the repertoire is diverse enough to be counted on to produce antibodies to “any” molecule a researcher may choose. This is, of course, an unreliable assumption, as experimental failures rarely find their way into the literature. The question that arises, therefore, is whether the immune repertoire is “complete” enough (15) to recognize and respond to the unprecedented geodesic structure of the fullerenes or sufficient aspects of it-more particularly, whether the immune system can process a fullerene-protein conjugate and display the processed peptides for recognition by T cells to yield IgG antibodies. We report here that it does.

### SUMMARY OF THE INVENTION

This invention provides two compositions. The first composition comprises a nanotube and at least one anti-nanotube antibody, wherein the anti-nanotube antibody is bound to the nanotube. The second composition comprises a fullerene and at least one anti-fullerene antibody, wherein the anti-fullerene antibody is bound to the fullerene.

This invention also provides seven methods using the two compositions. The first is for introducing the first and second compositions into a cell comprising contacting the composition with the cell under conditions permitting entry of the composition into the cell.

The second method is for determining whether an agent is present in a sample comprising contacting the sample with the composition comprising the antibody which has a moiety bound thereto, wherein the moiety of the composition permits the detection of the agent, and detecting any agent present in the sample via the moiety, thereby detecting whether the agent is present in the sample.

The third method is for introducing a moiety into a sample comprising introducing into the sample the composition comprising the antibody which has a moiety bound thereto, wherein the moiety being introduced into the sample is the moiety of the composition.

The fourth method is for immobilizing a nanotube on a solid support comprising contacting the first composition with a solid support having affixed thereto an agent that binds to the antibody of the composition, under conditions permitting such binding, thereby immobilizing the nanotube.

The fifth method is for immobilizing a nanotube on a solid support comprising contacting the first composition comprising the antibody which has a moiety bound thereto with a solid support having affixed thereto an agent that binds to the moiety of the composition, under conditions permitting such binding, thereby immobilizing the nanotube.

The sixth method is for immobilizing a fullerene on a solid support comprising contacting the second composition with a solid support having affixed thereto an agent that binds to the antibody of the composition, under conditions permitting such binding, thereby immobilizing the fullerene.

The seventh method is for immobilizing a fullerene on a solid support comprising contacting the second composition comprising the antibody which has a moiety bound thereto with a solid support having affixed thereto an agent that binds to the moiety of the composition, under conditions permitting such binding, thereby immobilizing the fullerene.

Finally, this invention provides five kits for using the two compositions. The first kit comprises the first and second compositions and instructions for use. The second kit comprises the composition comprising the antibody which has a moiety bound thereto and instructions for use. The third kit comprises a nanotube, an anti-nanotube antibody, and instructions for making and/or using the first composition. The fourth kit comprises a fullerene, an anti-fullerene antibody, and instructions for making and/or using the second composition. The fifth kit comprises the first and second compositions, a moiety, and instructions for binding the moiety to the antibody of the compositions.

### BRIEF DESCRIPTION OF THE FIGURES

FIG. 1

This figure shows fullerene derivatives used in this study.

FIG. 2

This figure shows the UV-Vis spectrum of 1-RSA and RSA, both at concentrations of 100 μg/ml in PBS.

FIG. 3

This figure shows the UV-Vis spectrum of 1-trilysine in water (80 μg/ml).

FIG. 4

This figure shows the ELISA study of antibody response to 1-RSA. White square, immune serum. Black diamond, preimmune serum.

FIG. 5

Results of double diffusion in agar. Well 1,1-TG (the immunogen); 2,2-TG; 3,1-BSA; 4,1-RSA; 5,3-RSA; and 6, TG.

FIG. 6

This figure shows the ELISA inhibition experiments: Black triangle, 1-BSA; open circle, 1-(Lys)₅; black circle, 1-RSA; open square, 1-TG; black square, 3-(Lys)₅; x, 2-(Lys)₅.

FIG. 7

Upper panel: This panel shows binding of the progesterone analog, 5-α-pregnane-20-one-3-β-ol hemisuccinate to the Fab′ fragment of a mAb specific for progesterone. This computer model was displayed in INSIGHT II from the x-ray crystal structure coordinates reported in reference 26.

Steroid is the dark cluster of spheres in the center at the top. Lower panel: This panel shows the molecular docking of fullerene-C₆₀ by deletion of the steroid and manual docking using INSIGHT II. Fullerene is the sphere in the center at the top.

FIG. 8

This figure shows computer models of fullerenes C₆₀ (7.2 A) and C₂₄₀ (−14 A). C₂₄₀ is the structure of the endcap of a [10, 10] single wall nanotube.

FIG. 9

This figure shows an electro microgram of micro-tubules with y-shaped MAB's bound to them along the side-walls.

These fibers are grown in-situ in response to the taxol-like MAB factors. The dimensions of micro-tubules are on the order of 25 nm, similar to multi-wall carbon nanotubes, although larger than single-wall nanotubes (SWNT).

FIG. 10

This figure shows the structure of a C82 fullerene containing an encapsulated atom.

FIG. 11

This figures shows the effect on weight gain of the fullerene derivative in Swiss mice (R. F. Schinazi et al. 1994).

FIG. 12

This figure shows the measured biodistribution of Ho@C₈₂(OH)ₓ metallo-fullerol.

FIG. 13

This figure shows fullerene derivatives to which anti-fullerene antibodies were produced by immunization of mice with a bovine thyroglobulin (TG) conjugate of a fullerene hemisuccinate F1 containing ca. 10-12 fullerenes per TG molecule. F3 is a C70 derivative.

FIG. 14

This figure shows the results of the double diffusion in agar experiment. Well 1 represents the FL-TG immunogen; 2, the F2-TG immunogen; 3, FL-BSA conjugate; 4, FL-RSA conjugate; 5, F3-RSA conjugate; and 6 is empty.

FIG. 15

This figure shows the ELISA inhibition test results.

FIG. 16

This figure shows the flow of the research studies (see infra “Fourth Series of Experiments”)

FIG. 17

This figure shows a mass spectrum (MS) of a typical sublimed sample (single step at high temperature) of Holmium (Ho) containing fullerenes is shown.

FIG. 18

This figure shows a purified Gd@C₈₂ using a single stage, anaerobic HPLC method applied to the high temperature sublimate.

FIG. 19

This figure shows an H-NMR spectrum of the hemisuccinate derivative of C_{θ0}.

FIG. 20

This figure shows the heavy chain sequence including leader of monoclonal antibody produced by mouse monoclonal anti-fullerene-hybridoma designated 1-10F-8A: 398 b.p. DNA sequence (SEQ ID NO:1) and encoded amino acid (SEQ ID NO:2).

FIG. 21

This figure shows the light chain sequence of monoclonal antibody produced by mouse monoclonal anti-fullerene-hybridoma designated l-10F-8A: 337 b.p. DNA sequence (SEQ ID NO:3) and encoded amino acid (SEQ ID NO:4).

FIG. 22

During their formation nanotubes acquire caps with fullerene geometries. This figure shows a rollup vector (n, m) that specifies the oriented width, according to the number of steps along the a and b directions. (Boris I. Yakobson and Richard E. Smalley, “Fullerene Nanotubes: C₁₀₀₀₀₀₀ and Beyond,” American Scientist, 85:324 (1997), hereby incorporated by reference).

FIG. 23

3-D crystal structure of the active site of a monoclonal antibody produced by the hybridoma produced by the fusion of a mouse antibody-producing cell and a mouse myeloma which is designated 1-10F-8A and deposited with the ATCC under Accession Number PTA-279, said hybridoma producing a monoclonal antibody which binds to fullerene C60.

FIG. 24

Panel a: This panel shows a tapping mode AFM height image (Digital Intruments NanoscopeIII) of SWNT on mica in air, data height range 15 nm. The SWNT ropes were obtained from Tubos at Rice Co. Panel b: This panel shows a height image of the same nanotube after exposure to fullerene-specific antibody for 8 min and washed with water, data range 15 nm. The tube shape has changed somewhat. Panel c: This panel shows a higher resolution surface plot of the tube in panel b, data range 15 nm. Panel d: This panel shows a surface plot of height image of SWNTs with fullerene-specific antibody on highly ordered pyrolytic graphite (HOPG), data range 20 nm.

FIG. 25

This Figure shows a schematic of a 2D graphene sheet illustrating lattice vectors a₁ and a₂, and the roll-up vector Cₕ=na₁+ma₂. The limiting, achiral cases of (n, 0) zigzag and (n, n) armchair are indicated with dashed lines. The translation vector T is along the nanotube axis and defines the 1D unit cell. The shaded, boxed area represents the unrolled unit cell formed by T and Cₕ. The diagram is constructed for (n, m)=(4, 2).

FIG. 26

This Figure shows an Atomic Force Micrograph of a nanotube with non-specific antibody (specificity was for Aldosterone receptor).

FIG. 27

This Figure shows an Atomic Force micrograph with anti-fullerene/anti-nanotube antibody bound to a nanotube (“U” shaped images represent the anti-fullerene antibody).

FIG. 28

This Figure shows an anti-fullerene/anti-nanotube antibody and nanotube in the presence of excess soluble fullerene (Compound 1 in Chen et al. PNAS (1998) 95:10809). Note the free antibodies in the field, displaced from the nanotube.

FIG. 29

This Figure shows the competitive inhibition by nanotube suspension. Specifically, this Figure quantifies the competition and shows that nanotubes compete very successfully with a fullerene compound for binding to an anti-fullerene antibody.

FIG. 30

This Figure shows the probe response to Ca⁺² ion concentration. Specifically, this Figure shows curves that show fluorescent (fluorescence) emission at 532 nm after exciting at 506 nm in the presence of varying concentrations of Ca⁺⁺. This Figure also shows that an antibody with probe C-3010 covalently attached can respond to Ca⁺². Legend:

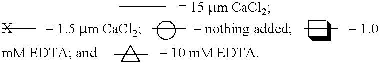

FIG. 31

This Figure shows the response of C-3010-labeled antibody to Ca⁺² concentration. Specifically, this Figure shows curves that show fluorescent (fluorescence) emission at 532 nm after exciting at 506 nm in the presence of varying concentrations of Ca⁺⁺. Legend (top panel):

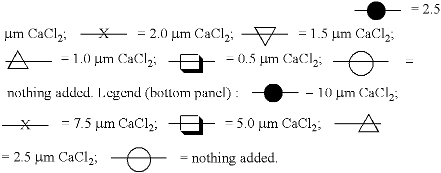

FIG. 32

This Figure shows the binding of Ca⁺² probe-antibody to C60-RSA. This figure shows that Ca⁺² probe-antibodies still bind fullerenes (attached to Rabbit serum albumin (RSA) as shown by ELISA). Legend:

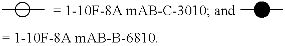

FIG. 33

This Figure shows that Ca⁺² probe modified antibodies bind nanotubes. Specifically, this Figure shows that an antibody decorated with a Ca⁺⁺ probe can bind nanotubes.

This is demonstrated by inhibition of antibody binding to fullerene-RSA by a colloidal suspension of nanotubes as determined by ELISA. Legend:

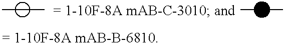

FIG. 34

This Figure shows the chemical structures of calcium ion probes, B-6810 and C-3010.

### DETAILED DESCRIPTION OF THE INVENTION

Definitions

As used in this application, except as otherwise expressly provided herein, each of the following terms shall have the meaning set forth below.

“Agent” shall mean any chemical entity, both organic and inorganic, including, without limitation, a glycomer, a protein, an antibody, a lectin, a nucleic acid, a small molecule, and any combination thereof.

“Amino acid,” “amino acid residue” and “residue” are used interchangeably herein to refer to an amino acid that is incorporated into a protein, polypeptide or peptide. The amino acid can be, for example, a naturally occurring amino acid or an analog of a natural amino acid that can function in a similar manner as the naturally occurring amino acid.

“Antibody” shall include, without limitation, (a) an immunoglobulin molecule comprising two heavy chains and two light chains and which recognizes an antigen; (b) a polyclonal or monoclonal immunoglobulin molecule; and (c) a monovalent or divalent fragment thereof. Immunoglobulin molecules may derive from any of the commonly known classes, including but not limited to IgA, secretory IgA, IgG, IgE and IgM. IgG subclasses are well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof. Antibodies may be human or nonhuman. Nonhuman antibodies may be humanized by recombinant methods to reduce their immunogenicity in humans. Antibody fragments include, without limitation, Fab fragments, Fv fragments and antigen-binding fragments.

As used herein, “humanized” describes antibodies wherein some, most or all of the amino acids outside the CDR regions are replaced with corresponding amino acids derived from human immunoglobulin molecules. Small additions, deletions, insertions, substitutions or modifications of amino acids are permissible as long as they do not abrogate the ability of the antibody to bind a given antigen. Suitable human immunoglobulin molecules include, without limitation, IgG1, IgG2, IgG3, IgG4, IgA and IgM molecules. Various publications describe how to make humanized antibodies, e.g., U.S. Pat. Nos. 4,816,567 (50), U.S. Pat. No. 5,225,539 (51), U.S. Pat. No. 5,585,089 (52) and 5,693,761 (53) and PCT International Publication No. WO 90/07861 (54).

“Anti-fullerene antibody” shall mean any antibody that binds a fullerene. In one embodiment, the anti-fullerene antibody binds a fullerene with higher affinity than any other molecule. In another embodiment the anti-fullerene antibody also binds a nanotube with equal or greater affinity than that to which it binds a fullerene.

“Anti-nanotube antibody” shall mean any antibody that binds a nanotube. In one embodiment, the anti-nanotube antibody binds a nanotube with higher affinity than any other molecule. In another embodiment the anti-nanotube antibody binds a fullerene with equal or greater affinity than that to which it binds a nanotube. Herein, the “anti-fullerene/anti-nanotube antibody” and “anti-nanotube/anti-fullerene antibody” shall each indicate an antibody which binds to both a nanotube and a fullerene, whether to equal or unequal degrees. Additionally, an anti-nanotube antibody can be generated using either a nanotube or a fullerene as an antigen. In one embodiment, binding occurs with a K_{D} of less than 10⁻⁵. In another embodiment, binding occurs with a K_{D} of less than 10⁻⁸. In a further embodiment, binding occurs with a K_{D} of less than 10⁻¹¹.

“Competitively inhibits” means that an antibody or fragment thereof competes with the monoclonal antibody produced by the hybridoma designated 1-10F-8A (ATCC Number PTA-279) for a binding site on a nanotube or on a fullerene.

As used herein, “detectable marker” includes, but is not limited to, a radioactive label, or a calorimetric, a luminescent, or a fluorescent marker. As used herein, “labels” include radioactive isotopes, fluorescent groups and affinity moieties such as biotin that facilitate detection of the labeled peptide. Other labels and methods for attaching labels to compounds are well-known to those skilled in the art.

“Discrete locus” shall mean a point, region or area for the affixation of a compound which does not overlap with another such point, region or area, and which may further be separated from another such point, region or area by physical space.

“Epitope” means an antigenic determinant present on a nanotube or on a fullerene and to which an antibody is capable of binding.

“Immobilized” shall mean attached by any means. In one embodiment, affixed shall mean attached by a covalent bond. In another embodiment, affixed shall mean attached non-covalently.

“Moiety” shall mean, unless otherwise limited, any chemical or biochemical entity. Examples of moieties include, without limitation, proteins (antibodies), nucleic acids, carbohydrates, small molecules and inorganic compounds.

The terms “nucleic acid”, “polynucleotide” and “nucleic acid sequence” are used interchangeably herein, and each refers to a polymer of deoxyribonucleotides and/or ribonucleotides. The deoxyribonucleotides and ribonucleotides can be naturally occurring or synthetic analogues thereof. “Nucleic acid” shall mean any nucleic acid, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA). Nucleic acids include, without limitation, anti-sense molecules and catalytic nucleic acid molecules such as ribozymes and DNAzymes. The nucleic acids of the subject invention also include nucleic acids coding for polypeptide analogs, fragments or derivatives which differ from the naturally-occurring forms in terms of the identity of one or more amino acid residues (deletion analogs containing less than all of the specified residues; substitution analogs wherein one or more residues are replaced by one or more residues; and addition analogs, wherein one or more residues are added to a terminal or medial portion of the polypeptide) which share some or all of the properties of the naturally-occurring forms.

The terms “polypeptide,” “peptide” and “protein” are used interchangeably herein, and each means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

“Sample”, when used in connection with the instant methods, includes, but is not limited to, any body tissue, skin lesion, blood, serum, plasma, cerebrospinal fluid, lymphocyte, urine, exudate, or supernatant from a cell culture.

“Solid support” includes, but is not limited to, any chip (e.g., silicone chip), slide (e.g., glass slide), filter, membrane, plate and bead. The solid support may be coated with, e.g., nitrocellulose, silicon or HYDROGEL. The use of these and other supports are known by one skilled in the art.

“Subject” shall mean any organism including, without limitation, a mouse, a rat, a dog, a guinea pig, a ferret, a rabbit and a primate. In the preferred embodiment, the subject is a human being.

Units, prefixes and symbols may be denoted in their SI accepted form. Unless otherwise indicated, nucleic acid sequences are written left to right in 5′ to 3′ orientation and amino acid sequences are written left to right in amino- to carboxy-terminal orientation. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Embodiments of the Invention

This invention provides an antibody which is specific for a single-walled fullerene nanotube. A nanotube (NT) is defined by the formula “[n, m]-nanotube” wherein n=0-500, m=0-500, and n and m are the same or different integers, i.e. [5,5]-NT (same), [9,0]-NT (different), but most commonly [10, 10]-NT (same). One of skill may refer for a more detailed description of nanotubes, for example, to Yakobson, B. I. and Smalley, R. E., American Scientist, 85:324 (1997) which is hereby incorporated by reference.

In an embodiment of the above-described antibody, the antibody is a monoclonal antibody. In another embodiment, the monoclonal antibody is a tip-specific antibody. In a further embodiment, the antibody is a side-wall specific antibody.

In an embodiment the above-described isolated nucleic acid is DNA or RNA. In another embodiment the isolated nucleic acid is cDNA or genomic DNA. In a further embodiment the encoded antibody has substantially the same amino acid sequences of the heavy chain (SEQ ID NO:2) and light chain sequences (SEQ ID NO:4) as set forth in FIGS. 20 and 21. In a still further embodiment, the nucleic acid encodes a human antibody. In another embodiment, the nucleic acid has nucleic acid sequences as set forth in FIGS. 20 and 21.

The DNA molecules of the subject invention also include DNA molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs wherein one or more amino acid residues is added to a terminal or medial portion of the polypeptides) and which share some or all properties of naturally-occurring forms. These molecules include: the incorporation of codons “preferred” for expression by selected non-mammalian hosts; the provision of sites for cleavage by restriction endonuclease enzymes; and the provision of additional initial, terminal or intermediate DNA sequences that facilitate construction of readily expressed vectors.

The DNA molecules described and claimed herein are useful for the information which they provide concerning the amino acid sequence of the monoclonal or polyclonal antibodies which are specific for single-walled fullerene nanotubes, or multi-walled fullerene nanotubes and as products for the large scale synthesis of the polypeptides (the monoclonal or polyclonal antibodies which are specific for single-walled fullerene nanotubes, or multi-walled fullerene nanotubes), or portions which are involved in protein-protein interactions by a variety of recombinant techniques. The molecule is useful for generating new cloning and expression vectors, transformed and transfected prokaryotic and eukaryotic host cells, and new and useful methods for cultured growth of such host cells capable of expression of the polypeptides (the monoclonal or polyclonal antibodies which are specific for single-walled fullerene nanotubes, or multi-walled fullerene nanotubes) or portions thereof and related products.

This invention provides an isolated antibody which specifically binds to both (a) a nanotube and (b) a fullerene selected from the group consisting of a fullerene having from 20 to 540 carbon atoms. In one embodiment, the fullerene comprises any one of the following: C60, C70, C76, C78, C84 and C240.

This invention provides a vector comprising the isolated nucleic acid which encodes a monoclonal or polyclonal antibody which is specific for single-walled fullerene nanotubes, or multi-walled fullerene nanotubes. In an embodiment(s) the vector(s) further comprises a promoter of RNA transcription operatively linked to the nucleic acid. In another embodiment of the above-described vectors the promoter comprises a bacterial, yeast, insect or mammalian promoter. In an embodiment the vectors may further comprise a plasmid, cosmid, yeast artificial chromosome (YAC), bacteriophage or eukaryotic viral DNA.

This invention provides a host vector system for the production of a polypeptide which comprises any of the above-described vectors in a suitable host. In an embodiment of the host vector system the suitable host is a prokaryotic or eukaryotic cell. In another embodiment of the host vector system the prokaryotic cell is a bacterial cell. In a further embodiment of the host vector system the eukaryotic cell is a yeast, insect, plant or mammalian cell.

Numerous vectors for expressing the inventive proteins may be employed. Such vectors, including plasmid vectors, cosmid vectors, bacteriophage vectors and other viruses, are well known in the art. For example, one class of vectors utilizes DNA elements which are derived from animal viruses such as bovine papilloma virus, polyoma virus, adenovirus, vaccinia virus, baculovirus, retroviruses (RSV, MMTV or MOMLV), Semliki Forest virus or SV40 virus. Additionally, cells which have stably integrated the DNA into their chromosomes may be selected by introducing one or more markers which allow for the selection of transfected host cells. The markers may provide, for example, prototrophy to an auxotrophic host, biocide resistance or resistance to heavy metals such as copper. The selectable marker gene can be either directly linked to the DNA sequences to be expressed, or introduced into the same cell by cotransformation.

Regulatory elements required for expression include promoter sequences to bind RNA polymerase and transcription initiation sequences for ribosome binding. Additional elements may also be needed for optimal synthesis of mRNA. These additional elements may include splice signals, as well as enhancers and termination signals. For example, a bacterial expression vector includes a promoter such as the lac promoter and for transcription initiation the Shine-Dalgarno sequence and the start codon AUG. Similarly, a eukaryotic expression vector includes a heterologous or homologous promoter for RNA polymerase II, a downstream polyadenylation signal, the start codon AUG, and a termination codon for detachment of the ribosome. Such vectors may be obtained commercially or assembled from the sequences described by methods well known in the art, for example the methods described above for constructing vectors in general.

These vectors may be introduced into a suitable host cell to form a host vector system for producing the inventive proteins (monoclonal or polyclonal antibodies specific for single-walled fullerene nanotubes, or multi-walled fullerene nanotubes). Methods of making host vector systems are well known to those skilled in the art.

Suitable host cells include, but are not limited to, bacterial cells (including gram positive cells), yeast cells, fungal cells, insect cells and animal cells. Suitable animal cells include, but are not limited to, HeLa cells, Cos cells, CV1 cells and various primary mammalian cells. Numerous mammalian cells may be used as hosts, including, but not limited to, the mouse fibroblast cell NIH-3T3 cells, CHO cells, HeLa cells, Ltk⁻ cells and COS cells. Mammalian cells may be transfected by methods well known in the art such as calcium phosphate precipitation, electroporation and microinjection.

One of ordinary skill in the art will easily obtain unique sequences from the cDNA cloned in plasmids. Such unique sequences may be used as probes to screen various mammalian cDNA libraries and genomic DNAs, e.g. mouse, rat and bovine, to obtain homologous nucleic acid sequences and to screen different cDNA tissue libraries to obtain isoforms of the obtained nucleic acid sequences. Nucleic acid probes from the cDNA cloned in plasmids may further be used to screen other human tissue cDNA libraries to obtain isoforms of the nucleic acid sequences encoding antibodies specific for single-walled fullerene nanotubes, or multi-walled fullerene nanotubes, as well as, to screen human genomic DNA to obtain the analogous nucleic acid sequences. The homologous nucleic acid sequences and isoforms may be used to produce the proteins encoded thereby.

This invention provides a method for producing a polypeptide which comprises growing any of the above-described host vector systems comprising the isolated nucleic acid which encodes any of the above-described polypeptides, i.e. antibodies specific for single-walled fullerene nanotubes, or multi-walled fullerene nanotubes under suitable conditions permitting production of the polypeptide(s) and recovering the polypeptide(s) so produced.

This invention provides a method of obtaining a polypeptide, i.e. an antibody specific for single-walled fullerene nanotubes, or multi-walled fullerene nanotubes in purified form which comprises: (a) introducing the vector comprising the isolated nucleic acid which encodes the polypeptide (antibody) into a suitable host cell; (b) culturing the resulting cell so as to produce the polypeptide; (c) recovering the polypeptide produced in step (b); and (d) purifying the polypeptide so recovered.

This invention provides a purified polypeptide which is an antibody specific for single-walled fullerene nanotubes, or multi-walled fullerene nanotubes. In an embodiment the above-described purified polypeptide has the amino acid sequences for the heavy and light chain sequences as set forth in FIGS. 20 and 21, respectively.

This invention provides an oligonucleotide of at least 15 nucleotides capable of specifically hybridizing with a unique sequence of nucleotides present within a nucleic acid which encodes an antibody specific for single-walled fullerene nanotubes, or multi-walled fullerene nanotubes. In an embodiment of the oligonucleotide of at least-1.5 nucleotides capable of specifically hybridizing with a unique sequence of nucleotides present within a nucleic acid which encodes an antibody specific for single-walled fullerene nanotubes, or multi-walled fullerene nanotubes, the isolated nucleic acids which encode the antibody have the nucleic acid sequences for the heavy and light chain sequences as set forth in FIGS. 20 and 21, respectively. In further embodiments of any of the above-described oligonucleotides the nucleic acid may be DNA or RNA.

This invention provides a nucleic acid having a sequence complementary to the sequence of the isolated nucleic acid which encodes an antibody which is specific for a single-walled fullerene nanotube, or a multi-walled fullerene nanotube, wherein the antibody is a monoclonal antibody or a polyclonal antibody.

This invention provides a polyclonal antibody which binds to a single-walled fullerene nanotube.

In an embodiment of the above-described polyclonal antibody which binds to a single-walled fullerene nanotube, the polyclonal antibody is a tip-specific antibody. In another embodiment, the antibody is a side-wall specific antibody.

This invention provides a polyclonal antibody which binds to a multi-walled fullerene nanotube.

All of the above described antibodies may be produced in a sheep, hog, goat, rabbit, guinea pig, mouse, rat and a human subject.

Polyclonal antibodies may be produced by injecting a host animal such as rabbit, rat, goat, mouse or other animal with the immunogen of this invention, e.g. a fullerene-protein conjugate, wherein the protein may be but is not limited to thyroglobulin, RSA, or BSA. The sera are extracted from the host animal and are screened to obtain polyclonal antibodies which are specific to the immunogen. Methods of screening for polyclonal antibodies are well known to those of ordinary skill in the art such as those disclosed in Harlow & Lane, Antibodies: a Laboratory Manual, (Cold Spring Harbor Laboratories, Cold Spring Harbor, N.Y.: 1988) the contents of which are hereby incorporated by reference.

The monoclonal antibodies may be produced by immunizing for example, mice with an immunogen. The mice are inoculated intraperitoneally with an immunogenic amount of the above-described immunogen and then boosted with similar amounts of the immunogen. Spleens are collected from the immunized mice a few days after the final boost and a cell suspension is prepared from the spleens for use in the fusion.

Hybridomas may be prepared from the splenocytes and a murine tumor partner using the general somatic cell hybridization technique of Kohler, B. and Milstein, C., Nature (1975) 256: 495-497. Available murine myeloma lines, such as those from the American Type Culture Collection (ATCC) 10801 University Blvd., Manassas, Va. 20110-2209, USA, may be used in the hybridization. Basically, the technique involves fusing the tumor cells and splenocytes using a fusogen such as polyethylene glycol. After the fusion the cells are separated from the fusion medium and grown in a selective growth medium, such as HAT medium, to eliminate unhybridized parent cells. The hybridomas may be expanded, if desired, and supernatants may be assayed by conventional immunoassay procedures, for example radioimmunoassay, using the immunizing agent as antigen. Positive clones may be characterized further to determine whether they meet the criteria of the invention antibodies.

Hybridomas that produce such antibodies may be grown in vitro or in vivo using known procedures. The monoclonal antibodies may be isolated from the culture media or body fluids, as the case may be, by conventional immuno-globulin purification procedures such as ammonium sulfate precipitation, gel electrophoresis, dialysis, chromato-graphy, and ultrafiltration, if desired.

In the practice of the subject invention any of the above-described antibodies may be labeled with a detectable marker. “Detectable markers” which function as detectable labels are well known to those of ordinary skill in the art and include, but are not limited to, a fluorescent label, a radioactive atom, a paramagnetic ion, biotin, a chemiluminescent label or a label which may be detected through a secondary enzymatic or binding step. The secondary enzymatic or binding step may comprise the use of digoxigenin, alkaline phosphatase, horseradish peroxidase, β-galactosidase, fluorescein or steptavidin/biotin. Methods of labeling antibodies are well known in the art.

Methods of encapsulating radioactive materials in fullerenes are known to one of skill in the art. For example Cagle, D. W. et al. (1999) PNAS (USA) 96:5182-5187 disclose the synthesis and use of radioactive metallofullerenes to monitor fullerene-based materials in vivo, wherein the radioactive material is holmium. One of skill in the art will recognize that radioactive materials other than holmium, e.g. gadolinium and other transition metals, may be encapsulated in fullerenes, including metallofullerenes.

This invention provides a method of preparing a nanoscale device which comprises manipulating a single-walled fullerene nanotube to assemble electronic or chemical components of the nanoscale device.

This invention provides a method of preparing a nanoscale device which comprises manipulating a multi-walled fullerene nanotube to assemble electronic or chemical components of the nanoscale device.

In an embodiment of any of the above-described methods of preparing a nanoscale device, the nanoscale device may be selected from the group consisting of a nanoscale optics device, a nanoscale electronic device, and a nanoscale biosensor device.

This invention further provides two compositions. The first composition comprises a nanotube and at least one anti-nanotube antibody, wherein the anti-nanotube antibody is bound to the nanotube. The second composition comprises a fullerene and at least one anti-fullerene antibody, wherein the anti-fullerene antibody is bound to the fullerene.

In one embodiment of the compositions, the antibody is a monoclonal antibody or an antigen-binding portion thereof. In one example of the compositions, the monoclonal antibody or antigen-binding portion thereof binds to the same epitope on the nanotube as the monoclonal antibody produced by the hybridoma designated 1-10F-8A (ATCC Number PTA-279). In another example, the monoclonal antibody or antigen-binding portion thereof competitively inhibits the binding to the nanotube of the monoclonal antibody produced by the hybridoma designated 1-10F-8A (ATCC Number PTA-279). In a further example, the monoclonal antibody or antigen-binding portion thereof is the monoclonal antibody produced by the hybridoma designated 1-10F-8A (ATCC Number PTA-279) or the antigen-binding portion thereof. In a further embodiment, the antibody is a polyclonal antibody.

In another embodiment of the compositions, the composition is immobilized.

In one embodiment of the first composition, the nanotube has bound thereto a plurality of anti-nanotube antibodies, and in the second composition, the fullerene has bound thereto a plurality of anti-fullerene antibodies.

In one embodiment of the first and second compositions, the compositions further comprise a moiety, wherein the moiety is bound to the antibody. The moiety can be a detectable marker, a probe, a small molecule, a polypeptide, an antibody or a nucleic acid. Detectable markers include, for example, a radioactive label, and a calorimetric, luminescent, or fluorescent marker. In one embodiment of the compositions, the probe permits the detection of ion concentration, such as Ca⁺² concentration. In such embodiment, the probe is C-3010 or B-8610.

This invention also provides seven methods for using the two compositions. The first method is for introducing the first and second compositions into a cell comprising contacting the composition with the cell under conditions permitting entry of the composition into the cell.

In one embodiment of the first method, the conditions permitting entry of the composition into the cell comprises the use of an atomic force microscope.

The second method is for determining whether an agent is present in a sample comprising contacting the sample with the composition comprising the antibody which has a moiety bound thereto, wherein the moiety of the composition permits the detection of the agent, and detecting any agent present in the sample via the moiety, thereby detecting whether the agent is present in the sample.

The third method is for introducing a moiety into a sample comprising introducing into the sample the composition comprising the antibody which has a moiety bound thereto, wherein the moiety being introduced into the sample is the moiety of the composition.

One skilled in the art would know how to practice the above methods for introducing moieties into samples. Various publications, describe how to introduce moieties into samples (44, 47-49 of “Sixth Series of Experiments” infra).

The fourth method is for immobilizing a nanotube on a solid support comprising contacting the first composition with a solid support having affixed thereto an agent that binds to the antibody of the composition, under conditions permitting such binding, thereby immobilizing the nanotube.

The fifth method is for immobilizing a nanotube on a solid support comprising contacting the first composition comprising the antibody which has a moiety bound thereto with a solid support having affixed thereto an agent that binds to the moiety of the composition, under conditions permitting such binding, thereby immobilizing the nanotube.

The sixth method is for immobilizing a fullerene on a solid support comprising contacting the second composition with a solid support having affixed thereto an agent that binds to the antibody of the composition, under conditions permitting such binding, thereby immobilizing the fullerene.

The seventh method is for immobilizing a fullerene on a solid support comprising contacting the second composition comprising the antibody which has a moiety bound thereto with a solid support having affixed thereto an agent that binds to the moiety of the composition, under conditions permitting such binding, thereby immobilizing the fullerene.

In one embodiment of the fourth, fifth, sixth and seventh methods, the solid support has the agent affixed thereto at one or more discrete loci.

Finally, this invention provides five kits for using the two compositions. The first kit comprises the first and second compositions and instructions for use. The second kit comprises the composition comprising the antibody which has a moiety bound thereto and instructions for use. The third kit comprises a nanotube, an anti-nanotube antibody, and instructions for making and/or using the first composition. The fourth kit comprises a fullerene, an anti-fullerene antibody, and instructions for making and/or using the second composition. The fifth kit comprises the first and second compositions, a moiety, and instructions for binding the moiety to the antibody of the compositions.

This invention will be better understood from the Experimental Details that follow. However, one skilled in the art will readily appreciate that the specific methods and results discussed are merely illustrative of the invention as described more fully in the claims which follow thereafter.

Experimental Details

First Series of Experiments

I. Materials and Methods

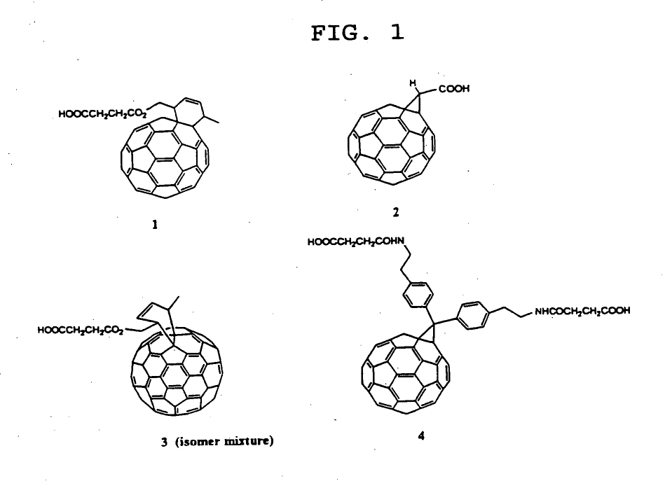

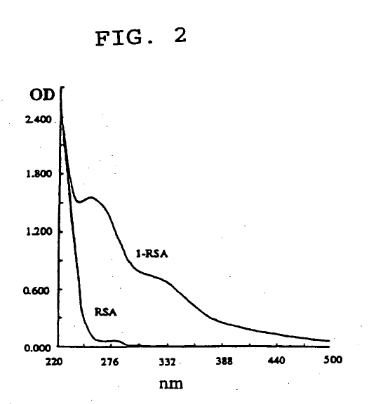

The fullerene derivatives 1-4 relevant to this invention are shown in FIG. 1. Compounds 1 and 3 were prepared as described in reference 16. For the synthesis of 2, see reference 17.

A. Preparation of the Bovine Thyroglobulin (TG) Conjugate of 1

Compound 1 (1.5 mg, 1.6 μmol) was dissolved in 0.25 ml of dry pyridine. N-Hydroxysuccinimide (Sigma) (8 mg, 70 μmol) was added and brought into solution with the fullerene compound. Dicyclohexylcarbodiimide (Fluka) (6 mg, 43 mmol) dissolved in 0.15 ml of dry pyridine was added, and the reaction was allowed to proceed at room temperature for 48 hours. The reaction mixture then was added dropwise over a period of about 5 minutes to 10.4 mg (1.3 μmol) of TG dissolved in 1 ml of water and adjusted to pH 9.5. The pH was kept at 8.5 throughout the reaction by the addition of 1 M NaHCO₃. Some turbidity appeared during the reaction. The reaction was allowed to proceed for 4 hours, and the reaction mixture then was dialyzed against PBS overnight at 4° C. The number of fullerene groups per molecule of TG was estimated, after clarification by centrifugation, to be ca. 20 by absorbance measurements at 320 nm (see below).

B. Bovine Serum Albumin (BSA) and Rabbit Serum Albwnin (RSA) Conjugates

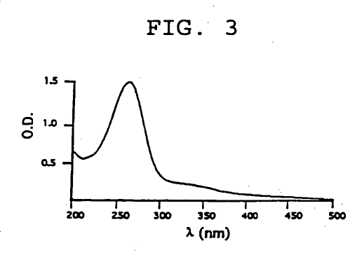

Similar procedures were used for the BSA and RSA conjugates. The UV-Vis spectrum of the RSA conjugate is shown in FIG. 2. It has a peak at 254 nm and a shoulder at about 320 nm. Others have seen these fullerene characteristics, albeit with slight shifts in wavelength (11, 16-19). The rise after 254 nm is characteristic of polypeptides, as shown by the spectrum of an equal concentration of RSA in FIG. 2. In both cases the proteins were substituted with about 10 molecules of the fullerene derivatives per molecule of protein, as determined by UV-Vis spectral analysis at 320 nm and by titration of the unsubstituted amino groups by trinitrobenzenesulfonic acid (20).

C. Conjugation of 1 to Lys-Lys-Lys.3HCl (3L)

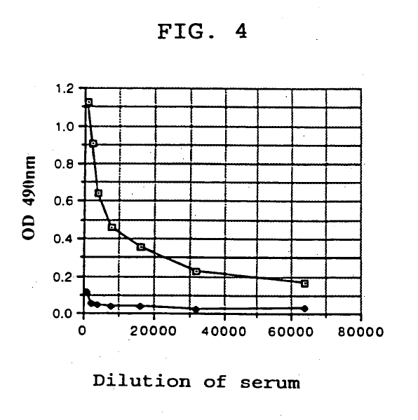

N-Hydroxysuccinimide (0.5 mg, 4.3 μmol) was dissolved in 0.125 ml of dry pyridine. The solution was added to 0.5 mg (0.54 μmol) of 1, which then was allowed to dissolve. Dicyclohexlycarbodiimide (2.5 mg, 18 μmol) was dissolved in 0.1 ml of dry pyridine, and the solution was transferred to the solution containing 1 and N-hydroxysuccinimide. The total reaction mixture was allowed to stand at room temperature for 48 hours. It then was added dropwise to 0.1 mg (0.2 μmol) of trilysine trihydrochloride (21) in 0.9 ml of 0.1 M NaHCO₃, with stirring. The pH was kept at 8.5 by addition of the bicarbonate solution. The reaction was allowed to proceed for 4 hours, and the resulting solution was taken to dryness over P₂O₅ in vacuo. The resulting residue was taken up in 250 μl of distilled water and clarified by centrifugation. Its UV-Vis spectrum is shown in FIG. 3.

Like 1-RSA, it has a shoulder and a peak, albeit somewhat shifted. The shoulder is at 335 nm and the peak is at 260 nm. From both its spectrum and reaction of its free amino groups with trinitrobenzenesulfonic acid (20) it was found to have an average of 1.8 mol of 1 per trilysine molecule.

D. Conjugation of 1 to Penta-L-Lysine (Sigma)

This reaction was carried out in a similar fashion as the trilysine conjugation. The final product was substituted to the extent of 2.7 mol of 1 per pentalysine.

E. Immunization Procedure and Detection of Antibodies

BALB/c mice were immunized i.p. with 1-TG in complete Freund's adjuvant for the primary immunization and incomplete adjuvant for subsequent immunizations.

After a total of three immunizations at 3-week intervals, confirmation of an immune response was determined by direct ELISA in which polystyrene plates (Corning) were coated with the 1-RSA conjugate (0.5 mg/ml in 0.1 M NaHCO₃, pH 9), and binding of preimmune and immune sera were determined by standard procedures. Development was with horseradish peroxidase-labeled goat anti-mouse IgG (Sigma), which tested negative for crossreaction with an IgM preparation. The substrate used was o-phenylenediamine.

Specificity of the response was determined by two procedures: double diffusion in agar (22) and competitive inhibition, as determined by ELISA using RSA and BSA conjugates of the fullerenes, as well as the oligo lysine derivatives. The fullerenes themselves were not soluble enough in aqueous solutions.

There were two controls for the ELISA experiments: (i) RSA, to show that the protein moiety did not participate in the inhibition, and (ii) adenosine-6-hexanoyl RSA (23), to show that the linkage group did not participate. The adenosine conjugate had been synthesized using the same strategy used for the fullerenes, i.e., linkage to the epsilon amino groups of the lysines via an N-hydroxysuccinimide ester derivative.

II. Results

A. Immune Response to 1-TG

The immune response of mice immunized with 1-TG first was determined by ELISA. Because of the extreme hydrophobicity of fullerenes, it was important to show that nonspecific binding to serum components did not occur in the preimmune serum. The results are shown in FIG. 4 for sera taken from a BALB/c mouse immunized i.p. with one primary and two booster injections 3 weeks apart. The result was a high titer of specific antibody, as measured with 1-RSA. No antibody or nonspecific binding was seen with components of the preimmune serum. Development was with a peroxidase-labeled anti-mouse antibody specific for mouse IgG.

B. Specificity of the Immune Response

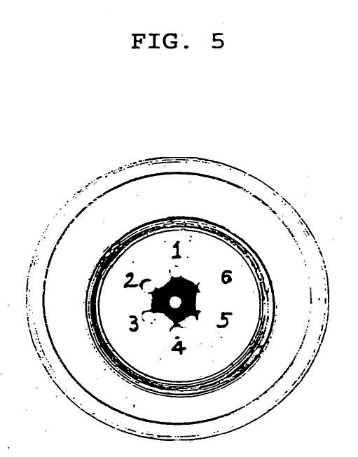

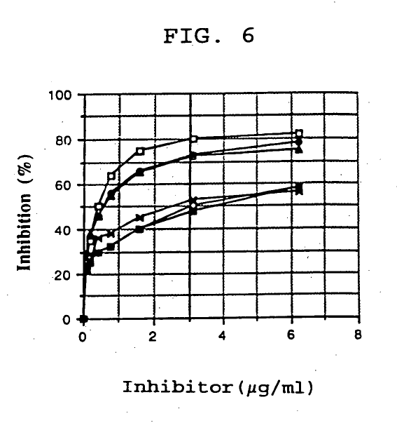

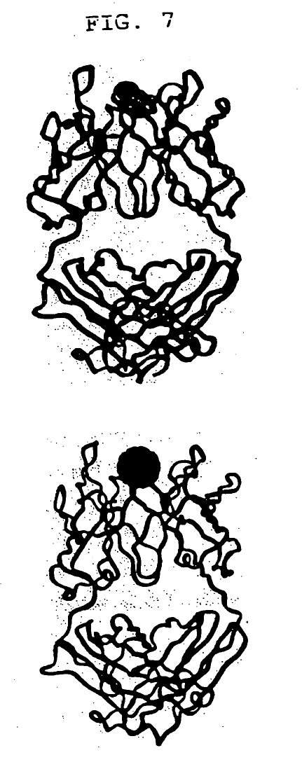

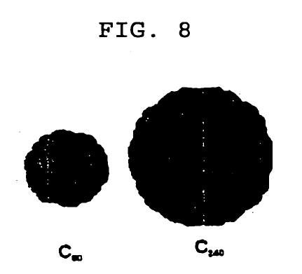

Specificity of the immune response first was determined by double diffusion in agar. The results are shown in FIG. 5.

Confluent lines of precipitation were seen associated with wells 1-5 with a spur between wells 2 and 3, pointing toward 3, i.e., toward 1-BSA. This finding is evidence of a population of antibodies reactive with both 0.1 and 2, with an additional population specific for the carrier protein of the immunogen, 1-TG. The identity of precipitation with 1 and 2 is evidence for the lack of participation of the linker group, which is missing from 2. A visible, albeit smaller, precipitate was seen with 3-RSA (well 5), the C₇₀ fullerene. Well 6, containing unsubstituted TG, showed no visible precipitate. However, when the TG solution was diluted 5-fold, a line of precipitation was seen (not shown), indicating a low titer of antibody specific for unsubstituted TG, i.e., in the original experiment (FIG. 5), i.e. TG was in antigen excess. Anti-TG also could be detected by ELISA (below).

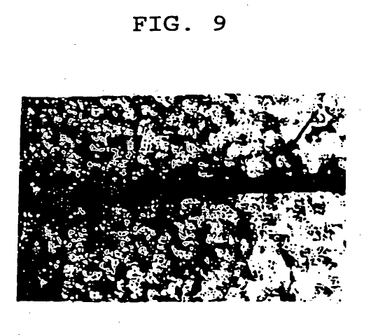

Specificity also was determined by ELISA inhibition studies (FIG. 6). Binding to 1-RSA was inhibited by 1-RSA, 1-BSA, 1-TG, 3-pentalysine, 2-pentalysine, and 1-pentalysine. Inhibition by 2-pentalysine is additional evidence for nonparticipation of the linker group, i.e., specificity for the C₆₀ moiety. Although not shown in FIG. 6 no inhibition was seen by TG, RSA or adenosyl-6-hexanoyl-RSA, which has the same linker joining the hapten to the protein carrier (23).

In an additional control for specificity, the fullerene derivatives 1-pentalysine, 1-RSA, and 1-BSA were tested by ELISA for nonspecific inhibition of an antibody to a retinoic acid derivative (unpublished work) with its hapten-RSA conjugate. No inhibition by the fullerene derivatives was seen, in contrast to the inhibition seen with the retinoic acid derivative (data not shown).

Taken together, the results show that there was an IgG response directed at the fullerenes, and not at the functional groups linking the fullerenes to the carriers. Moreover, a subpopulation of the antibodies raised to a C₆₀ fullerene crossreacted with the C₇₀ fullerene.

III. Discussion

Immunization of mice with a C₆₀ fullerene-TG conjugate produced a polyclonal response comprised of antibodies specific for C₆₀ fullerenes and a subpopulation that crossreacted with a C₇₀ fullerene derivative (FIGS. 5 and 6). Detection was possible by ELISA using an IgG-specific second antibody showing that the antibodies raised were of the IgG isotype (FIGS. 4 and 6). It follows from this that derivatization of TG by a fullerene molecule did not prevent intracellular processing and subsequent peptide display to T cells presumably by the process of linked recognition (24). Of interest is the manner of recognition of fullerenes by the immune system. Until we are in the position to examine the immune complex by direct means, e.g., x-ray crystallographic analysis, we can speculate based on characteristics of fullerenes that could provide potential for recognition.

A. Hydrophobicity

Fullerenes, being made up solely of carbon atoms, are very hydrophobic. It would be expected, therefore, that antibodies that recognize fullerenes will have hydrophobic amino acids in their binding sites. Such has been reported for the combining site of a Fab′ fragment of a mAb specific for progesterone (25, 26), a molecule highly apolar in character (see below). Contact with amino acids in the combining site of the heavy chain included three tryptophans, one tyrosine, and a phenylalanine in a buried combining site of 254 Å² in surface area (26).

B. Curvature

Theoretical studies of various fullerenes show that curvature, expressed as the pyrimidalization angle P, significantly influences fullerene properties and reactivity (27). Although the completely flat graphite molecule has a P angle of 0°, the most curved fullerene, C₆₀ has angles uniformly bent at 11.6°. The angles of the C₇₀ molecule vary from P=8.80 to almost 120 (see FIG. 1 for its shape). Curvature of a normally planar aromatic ring induces local charge differences.

C. pi Stacking

The pi system of fullerenes would be expected to interact with molecules in a combining site of an antibody via pi-stacking interactions. Experimental data from the x-ray structure of a C₆₀/benzene solvate clearly show this kind of interaction (28). Three benzenes are associated with each C₆₀ at distances of 3.27, 3.24, and 3.31 Å and are localized over the electron-rich interpentagon bonds. The C₆₀ molecule is not rapidly “jumping” from one orientation to another as observed in the unsolvated C₆₀ by neutron diffraction structure (29).

D. Uneven Charge Distribution

Although the charge distribution of C₆₀ is necessarily uniform, the charge distribution of a mono-substituted C₆₀ derivative such as 1 (FIG. 1) can show perturbations in its electron distribution (30). Moreover, it has been observed that C₆₀ and other fullerenes interact with donor —NH₂ and —SH groups (31, 32).

E. Combining site fit

We referred earlier to the interaction between progesterone and specific mAbs. The dimensions of C₆₀ and progesterone are very similar. Progesterone is longer and somewhat narrower (5.8 Å by 13 Å) (26); C₄₀ is a uniform sphere, 7.2 Å in diameter. The overall surface area, however, is very similar. There is no question about the “fit” of a fullerene in the combining site of an antibody.

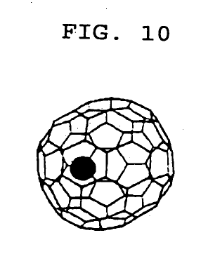

FIG. 7 (upper panel) shows the X-ray structure of an Fab′ fragment of a monoclonal progesterone-specific antibody bound to 5α-pregnane-20-one-3β-ol-hemisuccinate (26). The protein (Brookhaven PDB code 2 DBL) is displayed as ribbons and the steroid as a space-filling model by using INSIGHT II (Molecular Simulations, San Diego, Calif.). The binding site is a large hydrophobic cavity lined with Trp, Phe, and Tyr groups. Using INSIGHT II, we replaced the steroid with C₆₀ to provide the model shown in FIG. 7 (lower panel). As we did not relax the geometry using molecular dynamics, the fit is tight, but a slight side-chain adjustment would provide a very good fit.

F. Solvent Displacement

The free energy released on removing a hydrophobic surface from contact with water has been shown to correlate well with binding constants (33). Molecular modeling, studies of 4 (FIG. 1), a competitive inhibitor of an HIV protease, removed 298 Å² of solvent exposure (33). This stabilization reaction was mainly caused by carbon-to-carbon contacts with hydrophobic residues at the enzyme's active site: Leu, Ile, Tyr, Trp, Pro, Gly, and Ala.

The question of the binding of C₆₀ and C₇₀ fullerenes to Fab′ or Fv fragments of monoclonal antifullerene antibodies will be answered by x-ray crystallographic studies.

Finally, as part of this investigation three fullerene peptide derivatives have been prepared that are highly water soluble and the UV-Vis spectra of two of them has been presented.

### REFERENCES FOR FIRST SERIES OF EXPERIMENTS

1. Kroto, H. W., Heath, J. R., O'Brien, S. C., Curt, R. F. & Smalley, R. E. (1985) Nature (London) 318: 162-163.
2. Kratschmer, W., Lamb, L. D., Fostiropoulos, K. & Huffman, D. R. (1990) Nature (London) 347 354-357.
3. Scrivens, W. A., Bedworth, P. V. & Tour, J. M. (1992) J. Am. Chem. Soc. 114: 7917-7919.
4. Yu. G., Gao, J., Hummelin,. J. C., Wudl, F. & Heeger, A. J. (1995) Science 270: 1789-1791.
5. Yacobson, B. I. & Smalley, R. E. (1997) Amer. Sci. 85: 324-337.
6. Jensen, A. W., Wilson, S. R. & Schuster, D. I. (1996) Biorg. Med. Chem. 4: 767-779.
7. Sijbesma, R., Srdanov, G., Wudl, F., Castoro, J. A., Wilkins, C., Friedman, S. H., DeCamp, D. L. & Kenyon, G. L. (1993) J. M. Chem. Soc. 115: 6510-6512.
8. Schinazi, R. F., Sijbesma, R., Srdanov, G., Hill, C. L. & Wudl, F. (1993) Antimicrob. Agents Chemother. 37: 1707-1710.
9. Chiang, L. Y., Lu, J. T. & Lin, J. T. (1995) J. Chem. Soc. Chem. Commun., 1283-1284.
10. Lu, L.-H., Lee, Y.-T., Chen, H.-W. Chiang, L. Y. & Huang, H.-C. (1998) Brit. J. Pharmacol. 123: 1097-1102.
11. Toniolo, C., Bianco, A., Maggini, M., Scorrano, G., Prato, M., Marastoni, M., Tomatis, R., Spisani, S., Palu, G. & Blair, E. D. (1994) J. Med. Chem. 37: 4558-4562.
12. Dugan, L. L., Turetsky, D. M., Du, C., Lobner, D., Wheeler, M., Almi, C. R., Shen. C. K.-F., Luh, T.-Y, Choi, D. & Lin, T.-S. (1997) Proc. Natl. Acad. Sci. USA 94: 9434-9439.
13. Jerne, N. K. (1974) Ann. Inst. Pasteur/Immunol 125: 373-389.
14. Rajewsky, K., Forster, I. & Kumano, A. (1987) Science 238: 1088-1094.
15. Langman. R. E. (1992) Int. J. Clin. Lab. Res. 22: 63-68.
16. Wang, Y., Cao, J., Schuster, D. I. & Wilson, S. R. (1995) Tetrahedron Lett. 36: 6843-6846.
17. Bensasson. R. V., Bienveue, F., Janot, J.-M., Leach, S., Seta, P., Zhao, H., Schuster, D. I. & Wilson, S. R. (1995) Chem. Phys. Lett. 248: 566-570.
18. Anderson, T., Nilsson, K., Sundahl, M., Westman, G. & Wennerstrom, O. J. (1992) J. Chem. Soc. Chem. Comm., 604-606.
19. Boulas, P., Kutner, W., Jones, M. T. & Kadish, K. M. (1994) J. Phys. Chem. 98: 1282-1287.
20. Snyder, S. L. & Sobocinski, P. Z. (1975) Anal. Biochem. 64: 284-288.
21. Erlanger, B. F. & Brand, E. (1951) J. Am. Chem. Soc. 73: 4026-4027.
22. Johnstone, A. & Thorpe, R. (1982) Immunochemistry in Practice (Blackwell Scientific, Oxford), pp. 122-125.
23. Ku, H. H., Cleveland, W. L. & Erlanger, B. F. (1987) J. Immunol. 139: 2376-2384.
24. Janeway, C. A., Jr. & Travers, P. (1996) Immunobiology (Garland, N.Y.), pp. 8:3-8:5.
25. Erlanger, B. F., Borek, F., Beiser, S. M. & Lieberman, S. (1959) J. Biol. Chem. 234: 1090-1094.
26. Arevolo, J. H., Hassig, C. A., Stura, M. J., Taussig, M. J. & Wilson, I. A. (1994) J. Mol. Biol. 241: 663-690.
27. Haddon, R. C. (1993) Science 261: 1545-1550.
28. Meidine, M. F., Hitchcock, P. B., Kroto, H. W., Taylor, R. & Walton, D. R. M. (1992) J. Chem. Soc. Chem. Comm., 1534-1537.
29. David, W. I. F., Ibberson, R. M., Mattewman, J. C., Prassides, K., Dennis, T. J. S., Hare, J. P., Kroto, H. W., Taylor, R. & Walton. D. R. M. (1991) Nature (London) 353: 147-149.
30. Hirsch, A., Lamparth, I. & Grosser, T. (1994) J. Am. Chem. Soc. 116: 9385-9386.
31. Skiebe, A., Hirsch, A., Klos, H. & Gotschy, B. (1994) Chem. Phys. Lett. 220: 138-140.
32. Subramanian, R., Boulas, P., Vijayshee, M. N., D'Souzaj, F. & Jones, M. D. (1994) J. Chem. Soc. Chem. Commun., 1847-1848.
33. Friedman, S. H., DeCamp, D. L., Sijbesma, R. P., Srdanov, G., Wudl, F. & Kenyon, G. L. (1993) J. Am. Chem. Soc. 115: 6506-6509.
   Second Series of Experiments

The conjugate, C₆₀-thyroglobulin, is prepared as described below in “Preparation of Polyclonal Antibodies Specific for Fullerenes”. In those studies, polyclonal IgG antibodies were elicited in mice. A major population showed specificity for the C₆₀ hapten; a subpopulation reacted with C₇₀. Therefore, hybridomas that produce monoclonal antibodies specific for C₆₀ fullerenes and others specific for C₇₀ fullerenes may be isolated. On the other hand, it is not clear that the reaction with C₇₀ was not just a crossreaction, i.e. a population of anti-C₆₀ antibodies that cross-reacted with C₇₀. This possibility is taken into account during the screening process.

Alternatively, antibodies specific for C₇₀ fullerenes may be obtained by immunizing with a C₇₀-thyroglobulin conjugate. Here, too, however, the possibility of C₆₀ and C₇₀ cross-specificity exists. One interest, in this case, is to obtain C₇₀ specificity. Screening for specificity is by ELISA using RSA conjugates of the C₆₀ and C₇₀ fullerene derivatives shown in FIG. 1 of the first series of experiments.

Immunization of BALB/c mice is according to the protocol used to obtain the polyclonal anti-fullerene antibodies (see above), with thyroglobulin as the carrier protein, i.e. 1-TG (see FIG. 1, “First Series of Experiments”, for structure of 1). Primary immunization is by subcutaneous injection in Freund's complete adjuvant, followed three weeks later by a booster injection in incomplete Freund's adjuvant. Usually two additional booster injections, two weeks apart, are sufficient to raise a sufficiently high titer of specific antibody (assayed against the 1-RSA by ELISA) to start the hybridoma protocol.

The mouse is sacrificed, its spleen removed and spleen cells fused with a non-producer mouse myeloma, P3×63-Ag8.613, using PEG 1000 according to the procedure of Sharon et al. (1). The cells are placed in 96 well plates and, after two weeks incubation at 37° C., the supernatants are assayed by ELISA for binding to 1-RSA. Cells in positive wells are subcloned twice by limiting dilution and positive clones are isolated and grown in 96 well plates, then 24 well and finally in flasks, being assayed along the way.

Specificities for C₆₀ fullerenes and cross reactions with C₇₀ fullerenes are determined by ELISA. The specific aim is to isolate antibodies to C₆₀ that do not cross react with C₇₀ compounds. Immunization may also be performed with C₇₀-TG and selection for monoclonal antibodies with C₇₀ specificity may be accomplished.

The possibility of covalent linkage between fullerenes and a specific monoclonal antibody (above) is raised and can be tested for that in a preliminary way as follows:

Incubate aliquots of monoclonal antibody with fullerene-trilysine for periods of 2 hours-24 hours. After dialysis against PBS for 24 hours to remove unbound fullerene, the aliquots are assayed for binding to fullerene-RSA by ELISA. Covalent linkage would be indicated by a decrease in binding with time of incubation with fullerene-trilysine. The control will be antibody incubated for the same periods of time, but in the absence of fullerene-trilysine. Of course, unambiguous evidence of covalent linkage will be provided by the proposed x-ray crystallographic studies.

I. Materials and Methods

A. Preparation of Fab′ and Fv Fragments of Monoclonal Antibodies

1. Fab′ Fragments

The specific monoclonal antibody is converted into a Fab′ fragment after purification on a DEAE-cellulose column in phosphate buffer, pH 8.0. The IgG antibody leaves the column at the void volume. This is followed by digestion with papain in the presence of mercaptoethanol at 37° C. (enzyme: substrate=1:100). The course of digestion is followed by SDS-gel electrophoresis. The Fab′ fraction is then purified on a DEAE-cellulose column, precipitated with (NH₄)₂SO₄ and dialyzed against 0.01M potassium phosphate, pH 7.0. This is essentially the procedure used by Mariuzza et al. (2) for the preparation of Fab′ from a monoclonal anti-lysozyme antibody. The Fab′ was used in X-ray crystallographic studies. The Fab′ purification may also be done by carboxymethyl cellulose column (Porter (1951) Biochem. J.)

Fv fragments

Fv fragments are most suitably produced by molecular biological procedures as a recombinant protein. The variable chains that make up the Fv fragment can best be prepared by PCR cloning with the appropriate primers and expression either in bacteria (E. coli) or in eukaryotic cells. Enzyme digestion (e.g., pepsin) has been found to be less reliable.

With respect to expression in E. coli, one of skill may use the procedure of Orlandie et al. (3). In this procedure, RNA is isolated from about 116 hybridomas using guanidinium isothiocyanate. This is followed by reverse transcription of total mRNA using an oligo(dT) primer and then amplification of the resulting cDNA by PCR, using degenerate oligonucleotides based on conserved regions at the 5′ end of V gene segments and the 3′ end of the J segments. This is then followed by cloning of the amplified V_{H} and V_{L} genes in the pUC19-based dicistronic vector pSW1. It is in frame with the signal sequence of pectate lyase to allow secretion into the endoplasmic space of E. coli. Sequencing of several clones is necessary to ensure that random mutations have not occurred. This procedure was used by Goldbaum et al. to prepare Fv fragments for preliminary x-ray analysis of Fv-Fv complex (4, and see below).

One of skill may also use the cloning procedure developed by Coloma et al. (5, 6). This procedure allows for expression in non-producer myeloma cell lines such as NS2 or P3×63-Ag8.653. Most of applicants' hybridoma experience is with the latter line and therefore, this procedure is preferred for expression of the Fv fragments.

B. The Fullerene Derivative Co-crystallized with the Fab′ and Fv Fragments of the Antibody

The synthesis of a trilysine derivative of the C₆₀ fullerene compound 1 has already been described (see below). This derivative is, however, a mixture with an average substitute of 1.8 fullerenes per trilysine. For the co-crystallization experiments, the monosubstituted derivative is preferred. The separation of the pure monosubstituted compound from the mixture can be accomplished by reverse phase or ion exchange chromatography because the various components of the mixture differ in hydrophobicity and charge.

C. Preparation of Polyclonal Antibodies Specific for Fullerenes

The fullerene derivatives 1-4 used in the studies relevant to this invention are shown in FIG. 1. Compounds 1-3 were prepared as described in reference 16, “First Series of Experiments”. For the synthesis of 2, see reference 17, “First Series of Experiments”.

D. Preparation of the Bovine Thyroglobulin (TG) Conjugate of 1

The conjugate was made according to the procedure set forth in the “First Series of Experiments”. The number of fullerene groups per molecule of TG was estimated after clarification by centrifugation to be ca. 20 by absorbance measurements at 320 nm (see below).

E. Bovine Serum Albumin (BSA) and Rabbit Serum Albumin (RSA) Conjugates

The conjugates were made according to the procedure set forth in the “First Series of Experiments”.

F. Conjugation of 1 to Lys-Lys-Lys.3HCl (3L)

N-Hydroxysuccinimide (0.5 mg; 4.3 μmoles) was dissolved in 0.125 ml of dry pyridine. The solution was added to 0.5 mg (0.54 μmoles) of 1, which was then allowed to dissolve. Dicyclhexlycarbomiimide (2.5 mg); 18 mmoles) was dissolved in 0.1 ml of dry pyridine and the solution transferred to the solution containing 1 and N-hydroxysuccinimide. The total reaction mixture was allowed to stand at room temperature for 48 hours. It was then added dropwise to 0.1 mg (0.2 μmoles) of trilysine trihydrochloride (21, “First Series of Experiments”) in 0.9 ml of 0.1 N NaHCO₃, with stirring. The pH was kept at 8.5 by addition of the bicarbonate solution. The reaction was allowed to proceed for 4 hours, and the resulting solution taken to dryness over P₂O₅ in vacuo. The resulting residue was taken up in 250 μl distilled water and clarified by centrifugation. Its UV-Vis spectrum is shown in FIG. 3, “First Series of Experiments”. Like 1-RSA, it has a shoulder and a peak, albeit somewhat shifted: The shoulder is at 335 nm and the peak is at 260 nm. From both its spectrum and reaction of its free amino groups with trinitrobenzenesulfonic acid (20, “First Series of Experiments”) it was found to have an average of 1.8 molecules of 1 per trilysine molecule.

G. Conjugation of 1 to penta-L-lysine (Sigma, St. Louis, Mo.)

This reaction was carried out in a similar fashion as the trilysine conjugation. The final product was substituted to the extent of 2.7 molecules of 1 per pentalysine.

H. Immunization Procedure and Detection of Antibodies

Balb/C mice were immunized i.p. with 1-TG in complete Freund's adjuvant for the primary immunization and incomplete adjuvant for subsequent immunizations.

After a total of 3 immunizations at 3 week intervals, confirmation of an immune response was determined by direct ELISA in which polystyrene plates (Corning) were coated with 1-RSA conjugate (0.5 mg/ml in O.1 N NaHCO₃, pH 9) and binding of preimmune and immune sera determined by standard procedures. Development was with horseradish peroxide-labeled goat anti-mouse IgG (Sigma, St. Louis, MO) which tested negative for cross reaction with an IgM preparation. The substrate used was o-phenylenediamine.

Specificity of the response was determined by two procedures: double diffusion in agar and competitive inhibition, as determined by ELISA using RSA and BSA conjugates of the fullerenes, as well as the oligo lysine derivatives. The fullerenes themselves were not soluble enough in aqueous solutions.

There were two controls for the ELISA experiments: 1) RSA, to show that the protein moiety did not participate in the inhibition; and 2) adenosine-6-hexanoyl RSA (23, “First Series of Experiments”), to show that the linkage group did not participate. The adenosine conjugate had been synthesized using the same strategy used for the fullerenes, i.e., linkage to the epsilon amino groups of the lysines via an N-hydroxysuccinimide ester derivative.

II. Results

A. The Immune Response to 1-TG

The immune response of mice immunized with 1-TG was first determined by ELISA. Because of the extreme hydrophobicity of fullerenes, it was important to show that non-specific binding to serum components did not occur in the pre-immune serum. The results are shown in FIG. 4 (“First Series of Experiments”) for sera taken from a Balb/C mouse immunized i.p. with one primary and two booster injections three weeks apart. The result was a high titer of specific antibody, as measured with 1-RSA. No antibody or non-specific binding was seen with components of the pre-immune serum. Development was with a peroxidase-labeled anti-mouse antibody specific for mouse IgG.

B. Specificity of the Immune Response

Specificity of the immune response was first determined by double diffusion in agar. The results are shown in FIG. 5 (“First Series of Experiments”). Lines of precipitation were seen associated with wells 1-5 with a spur between wells 2 and 3, pointing toward 3, i.e. toward 1-BSA. This implies an additional reactive epitope in 1-TG, not present in the BSA conjugate. A visible, albeit small precipitate was seen with 3-RSA (well 5). Well 6, containing unsubstituted TG showed no visible precipitate. However, when the TG solution was diluted five-fold, a line of precipitation was seen (not shown), indicating a low titer of antibody specific for unsubstituted TG., i.e. in the original experiment (FIG. 5, “First Series of Experiments”) TG was in antigen excess. Moreover, anti-TG could be detected by ELISA (below).

Specificity was also determined by ELISA inhibition studies (FIG. 6, “First Series of Experiments”). Binding to 1-RSA was inhibited by 1-RSA, 1-BSA, 1-TG, 3-pentalysine and 1-pentalysine. No inhibition was seen by TG, RSA, or adenosyl-6-hexanoyl-RSA, which has the same linker joining the hapten to the protein carrier (23, “First Series of Experiments”). As an additional control for specificity, 1-RSA, 1-pentalysine and 1-BSA were tested by ELISA as non-specific inhibitors of an antibody to retinoic acid (unpublished). No inhibition was seen, in contrast to inhibition by retinoic acid.

### REFERENCES FOR SECOND SERIES OF EXPERIMENTS

1. Sharon, J. M. et al., (1983) Proc. Natl. Acad. Sci. USA, 76: 1420-1424.
2. Mariuzza, R. A. et al. (1983) J. Mol. Biol. 170: 1055-1058.
3. Orlandi, R. et al. (1989) Proc. Natl. Acad. Sci. USA, 86: 3833-3837.
4. Goldbaum, F. A., et al. (1994) J. Mol. Biol., 241: 739-743.
5. Leu, J.-G., et al. (1994) Proc. Natl. Acad. Sci. USA, 91: 10690-10694.
6. Coloma, M. J., et al. (1992) J. Immunol. Meth. 152: 89-104.
7. Jerne, N. K. et al., (1974) Ann. Immunol. (Inst. Pasteur) 125C: 373-389.
8. Cleveland, W. L., et al. (1993) Nature 205: 56-57.
9. Erlanger, B. F., et al. (1991) Biochem. Soc. Transactions., 19:138-143.
10. Braden, B. C., et al. (1996) J. Mol. Biol. 264: 137-151.
11. Braden, B. C., et al. (1998) The Immunologist, 6: 13-18.
   THIRD SERIES OF EXPERIMENTS

The objective of these studies is to prepare and screen antibodies to carbon clusters and nanotubes: fullerene compounds C60, C70, C240 and [10, 10]-nanotubes. It is expected that C240-specific monoclonal antibodies (MAB's) can be used to recognize the tips and/or side walls of single wall nanotubes (SWNT's). The interactions of carbon clusters and nanotubes with these biomolecules by a variety of means, including protein X-ray crystallography are to be characterized. MAB's to SWNT's may be used to develop assays that will accelerate SWNT processing, applications, and commercialization. Finally, basic science issues involving the future use of antibodies as assemblers are explored that will allow programmed assembly of nanotubes at the nanometer and mesoscopic scale.

Fullerenes and Nanotubes

The discovery of a new form of carbon fullerene-C60 in 1985 by Smalley and coworkers at Rice University stimulated tremendous world-wide research interest (1). When the Huffman-Kratschner (HK) arc process for production of fullerene-C60 in quantity made samples of C60 available in 1991, scientists throughout the world began to explore C60 chemistry. Close relatives of C60, fullerene nanotubes (very long graphitic molecular fibers), were first observed in the HK arc process by Iijima (2). Numerous theoretical and microscopic studies of nanotubes followed this exciting discovery (3). However, it was the recent breakthrough by Smalley—preparation of gram quantities of fullerene single wall nanotubes (SWNT)—that promises to propel SWNT research to commercial application. SWNT's are called “the perfect carbon fibers.” They conduct electricity as well as gold, and are 100× stronger than steel at ⅙ the density.

Important DoD-related applications include mechanical/chemical applications as well as optical/electronic applications. Possible mechanical/chemical uses are lightweight, high strength composites for vehicle body panels, ship hulls and airframes, ship and helicopter propellers, rocket nozzles, helmets and body armor. Possible electronic/optical applications include tactical displays, laser eye protection, vacuum electronics, capacitors, batteries and fuel cells. Other uses may include chemical filters, catalyst supports, hydrogen storage and nanoscale devices for computation (4).

The enormous promise of nanotubes requires the development of many supporting basic science areas to assist in characterization and to begin evaluation of potential biological applications and potential health risks. Considerable work is known about C60 chemistry and much work has been done on toxicology and even drug development with fullerenes (5). The toxicology of large carbon fibers has been extensively studied and a review is available (6). Nothing at all is known about toxicology of nanotubes or their interactions with biological systems. While no general toxic effects of fullerenes are known, the small diameter and very large aspect ratio (ratio of length to diameter) of nanotubes is somewhat reminiscent of asbestos fiber, and make an important side-benefit of this research the beginning first steps to the understanding the relationship of nanotubes to human health.

Antibodies

While there is not enough space here to even begin to review the field of immunology, it can be simply pointed out that cells of the immune system can produce such antibodies in response to foreign molecules called antigens. Antibodies are protein molecules of molecular weight—150,000 and are created by antibody-producing cells to bind strongly to an antigenic foreign molecule. Antibodies produced directly by animal immunization are polyclonal since they are produced by a multiplicity of cells that respond to the presence of the antigen. Techniques are also available for the preparation of monoclonal antibodies (MAB). MAB's are prepared from a single cloned cell line and thus only one pure molecular form of the antibody is obtained. MAB's are desirable since they are pure molecules, can be crystallized, and molecular biological techniques be used to sequence the gene of the MAB, study, characterize, model, and modify the structure, and prepare crystals for X-ray crystallography.

The development of methods for the preparation of antibodies to hydrophobic small molecules is well known to one of skill in the art (7). During the 1950's and 60's Erlanger developed the approach of linking steroid molecules to serum albumins, to immunize rabbits with the steroid-serum albumin conjugate, and to thereby obtain specific anti-steroid antibodies. This method has revolutionized detection of hydrophobic compounds in biological systems. Erlanger has prepared monoclonal antibodies to the hydrophobic molecule taxol as well as a MAB that mimics taxol (8).

Approach and Preliminary Studies

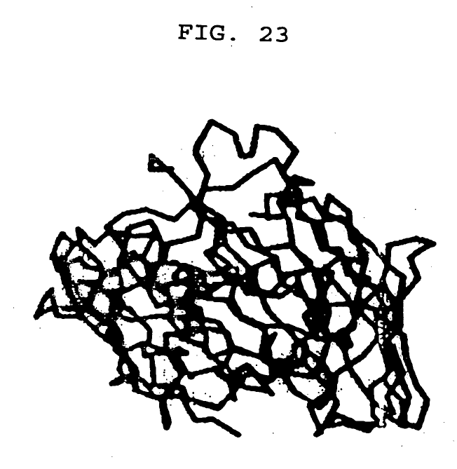

Fullerene and nanotube antibodies are to be prepared and MAB to selected fullerenes have been crystallized. It is possible to produce X-ray quality crystals and to determine the complete 3D structure of these antibodies (see, for example, FIG. 23 which shows the 3D structure of the active site of an anti-fullerene monoclonal antibody). Antibodies to nanotubes consist of two classes: tip-specific antibodies and side-wall specific antibodies. Tip-specific antibodies are prepared using antibodies produced using a related fullerene hapten. The tips of [10, 10]-SWNT's are predicted to be hemi-fullerenes, specifically one half of a C240 molecule. Although C240 has been detected by mass spectrometry and STM, it has not yet been isolated (8, 9). Vigorous work in that area is continuing. Currently only C60, C70, C76, C78, and C84 are available in commercial quantities.

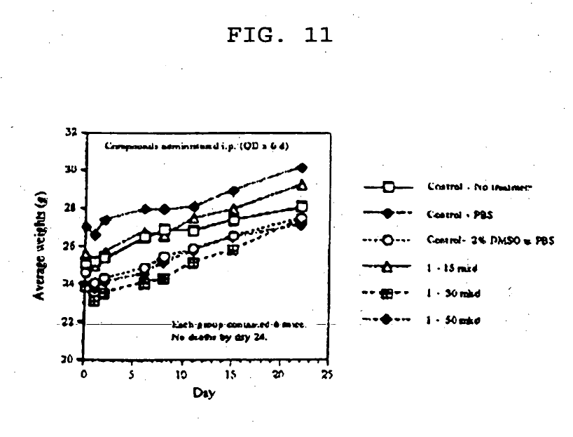

While C240 is much larger that C60 (see FIG. 8) it is well within the molecular scale of known antibody combining sites. An antibody to dextran binds 5 glucose residues which corresponds to a combining sites 2.5 nm long. (The taxol anti-idiotypic antibody shown in FIG. 2 and discussed later binds to tubes 25 nm in diameter)(8).

Considerable chemistry is now known about C60, and so the first experiments used this simplest fullerene. Preliminary studies are very encouraging and indicate that excellent polyclonal antibodies can be prepared by essentially conventional methods. Fullerene C60 was treated as a hapten and covalently linked to bovine serum albumin (BSA) and rabbit serum albumin (RSA) in order to induce a T-dependent immune response which can lead to high affinity antibodies. Linkage to BSA and RSA was performed via an N-hydroxysuccinimide ester derivative of C60. A rabbit was immunized with BSA-fullerene conjugate dissolved in aqueous solution and emulsified with Freund's adjuvant. This mixture was injected intradermally in multiple regions of the back of a rabbit. This initial immunization was followed by three booster immunizations. Serum drawn from the rabbit was tested for fullerene-specific antibodies using the RSA conjugate of fullerene-C60. This was done because the rabbit does not make antibodies to its own protein, RSA.

Hence any reaction would be directed at the fullerene-C60 moiety. The antibodies were detected using an ELISA technique. Recognition of the water soluble anti-viral fullerene derivative, originally synthesized by Wudl, by the C60 specific antibodies has been demonstrated.

The goal of these studies is to develop methods to prepare high-affinity monoclonal antibodies to fullerenes and carbon nanotubes and to characterize their interactions by a variety of means including X-ray crystallography. This work explores the application of antibodies to manupulation of nanotubes.

The long range goal of this invention is to design an antibody-based nanotube analysis system and create a nanoassembler system that could assist in the organization of SWNT's into useful nanoscale devices. Fullerene antibodies serve as the basis for an important enabling science—connecting the “dry-side” of nanotechnology with the “wet-side”, i.e. biology.

Initially prepared and characterized are the antibodies to fullerene-C60 and to SWNT's. This enables one to learn more about how best to link, solubilize, and characterize antibodies to carbon clusters and SWNT's and to develop new methods to solve fundamental problems in analysis of SWNT structures, for example, to distinguish between [10, 10] tubes and [n,m] tubes using specific antibodies.

Technology to use monoclonal antibodies (MAB) to allow assembly and organization of carbon clusters and SWNT's into large mesoscopic arrays producing useful devices are also encompassed within the scope of the present invention. This use is widely promoted in nanotechnology, and may be possible by harnessing monoclonal antibody technology to do it. A good demonstration has already ben accomplished. Cell division (mitosis) is mediated by a complex tubulin by the mediation of a small hydrophobic molecule called Taxol. Erlanger has shown this process can also be carried out by an anti-idiotypic antibody which mimics the action of taxol and stimulates the process of micro-tubule organization as shown in FIG. 2. It is suggested that tip-specific and side-wall specific SWNT MAB's of various sorts may be developed as “nano-assemblers” for the arrangement of SWNT-based devices. It may be possible to reconstitute catalytic MAB's that operate in organic media to promote the assembly and orientation of SWNT's into useful nanoscale devices on the surface of silicon chips (11).

FIG. 9 shows an electro-microgram of micro-tubules with γ-shaped MAB's bound to them along the side-walls. Theses fibers are grown in situ in response to the taxol-like MAB factors. The dimensions of micro-tubules are on the order of 25 nm, similar to multiwall carbon nanotubes, although larger that SWNT.

### REFERENCE FOR THIRD SERIES OF EXPERIMENTS

1. “Buckminsterfullerene,” Billips, W. E., Clufolini, M. A., Eds, VCH, NY (1992).
2. “Single-shell carbon nanotubes of 1-nm in diameter,” Iijima, E., Ichihashi, T. Nature 361:603 (1993).
3. “Carbon Nanotubes,” Ann. Rev Material Sci., 243: 235 (1994).
4. “Fullerene Nanotubes: C1,000,000 and Beyond,” Yokabsen, B. I., Smalley, R. E. American Scientist, 85: 324 (1997).
5. “Biological Applications of Fullerenes-A Review,” Jenson, A., Wilson, S. R., Schuster, D. I., Bioorganic and Medicinal Chemistry, 4: 767 (1996).
6. “Toxicology of Carbon Fibers,” Thomson, S. A. Appl. Ind. Hyg. 29 (1989).
7. “Preparation of Antigenic Hapten-Carrier Conjugates,” Erlanger, B. F. Methods in Enzymology 70: 85 (1980).
8. “Idiotypic mimicry and the assembly of a supramolecular structure; and anti-idiotypic antibody that mimics taxol in its tubulin-microtubule interactions,” Leu, J-G, Chen, B-X, Diamanduras, A. W., Erlanger, B. Proc. Natl. Acad. Sci. USA 91: 10690 (1994).
9. “Mass spectrometry and STM Imaging of Giant Fullerenes,” Lamb, L. D. et. al. Science 255: 1413 (1992).
10. “C240-the least reactive Fullerene,” Haddon, R. C.; Scusaria, G. E., Smalley, R. E. Science (in press).
11. “Enzymic Catalysis in Anhydrous Organic Solvents,” Klibanov, A. M. Trends Biochem Sci 14: 145 (1989).
   Fourth Series Experiments
   I. Background
   Radioimmunotherapy (RIT) has the potential to deliver doses of highly energetic radiation specifically to cancerous growths, thereby eliminating the radioactive damage to healthy tissue common during externally delivered radiation therapy. The traditional method for RIT is to link a radioactive atom to a monoclonal antibody (mAb) specific for the tumor, and inject it into the patient. The mAb then circulates until it binds to the tumor, where the radionuclide decays, emitting radiation to destroy the cancer cells. To date, the radioactive metal atoms have been bound to a chelating molecule, which is in turn linked to the mAb specific to the epitope on the tumor. Similar methods have been developed for radioimmunoimaging (RII), where a gamma ray emitter is bound to the chelating compound, and scintography is used to detect the concentrated areas of gamma rays which mark the tumor.

Unfortunately, in the methods developed to date, almost none of the administered radioactive dose reaches the cancer. As a result, clinical trials have shown poor response rates to RIT against solid malignancies (Wilder et al., 1996). In conventional methods, the primary problem is the long time required for the mAb to find the cancer cells. While the mAbs circulate, a significant fraction of the radionuclides decay, damaging healthy tissue and resulting in low tumor/nontumor (T:NT) ratios of radioactivity. Further, the linker can be metabolized, separating it from the mAb, and/or the metal can become unchelated, both of which release radionuclides that will never reach the tumor.

Complete containment of the radionuclide can be achieved by using endohedral metallofullerenes (FIG. 10). Fullerenes are the third allotrope of carbon, a class of nearly spherical cages roughly one nanometer in diameter. The high strength of the carbon SP2 bonds and durability of the fullerenes ensure that the encapsulated metal atom remains in the cage, even under beta recoil energies in excess of 10 eV (Kikuchi et al., 1994). The cage can be functionalized to become water-soluble without loss of integrity. Since the metal cannot escape a fullerene cage, isotopes with longer half-lives can reduce background radiation doses by allowing the mAb more time to find the cancer before decay occurs. These properties make fullerenes an attractive option to replace chelators for the delivery of radionuclides.

Multi-step targeting has shown promise as a means of increasing the T:NT ratio. The first injection in these methods is a bispecific antibody, where one end binds to the tumor and the other is specific for the chelating agent. The chelated radionuclide is injected later, after the bispecific antibody has achieved its maximum localization on the tumor. These methods suffer from the same problems that release radionuclides, but for a shorter time. Since the advantage is gained by using more rapidly circulating (and clearing) radiolabeled molecules, the smallest molecule that can firmly chelate the radionuclide and be specific for its receptor is desirable. In these respects, metallofullerenes are also ideal for radionuclide delivery in multi-step targeting.

Very recently, antibodies to empty fullerenes were developed by molecular bioengineering, providing a receptor for the metallofullerene. These new molecular tools prompted us to design an improved method for administering RIT. The radioisotope-containing water-soluble fullerene is administered after a bispecific antibody, where one end is the fullerene antibody (Abf) and the other is the tumor-specific mAb, has achieved its maximum localization on the tumor. Since the binding strength of the essentially hydrophobic fullerene to its own antibody can be very high, and water-soluble fullerenes will circulate rapidly without strong retention in any particular organ, this is a promising way to improve the T:NT ratio.

This invention shows that the metallofullerene Ho@C₈₂ (one holmium atom inside of a carbon cage containing 82 carbon atoms) can be derivatized to become water-soluble, and then bound to the bioengineered Abfs. Ho@C₈₂ is bound to a monoclonal antibody bioengineered specifically for C₈₂ containing a metal atom. The available Abf at present is polyclonal produced in response to C₆₀, the most abundant fullerene. It is non-reactive with C₇₀, but not as yet been tested with C₈₂. The C₈₂ fullerene has been chosen as a host for the lanthanide because methods for its purification are much better developed than for other endohedral metallofullerenes. Virtually all lanthanides can be encapsulated with equal ease: holmium was selected because therapeutic applications of the ¹⁶⁶Ho isotope have already been proposed. The process of neutron activating (Ho@C₈₂)R (where R is a group inducing water solubility) to become (166Ho@C₈₂)R is currently being studied. The attachment of metallofullerenes to antibodies is also studied.

Metallofullerene-based RIT treatments is one long term goal of these studies and precedents for mAb-based cancer therapies have already been established. For example, the mAb B72.3, developed at the National Cancer Institute, has been approved by the FDA and licensed by the NIH to pharmaceutical companies. Cytogen Corp. employs it as a basis for diagnostic agents for the detection and imaging of colo-rectal and ovarian cancers. More advanced, second generation antibody technology (e.g., CC49) is also becoming available and is currently licensed for RII and RIT applications. Bispecific antibodies, such as MDX-210 (Medarex, Inc.) and 2B1 (Chiron Corp.), are also receiving approval for cancer therapies involving a triggering of the patient's own immune system.

Endohedral fullerenes and their produced derivatives may be purified and used in the methods developed in this invention. Endohedral fullerenes are predicted to exhibit many unique properties that make them potentially valuable commodities. As an example, calculations predict that C₈₀ containing trapped lanthanide ions will be a high temperature superconductor.

With respect to medical markets, all applications involving the transport of metals in vivo can benefit from metallofullerenes. Ho@fullerene based materials may be used as radionuclide carriers and a specific use as labeling agents. Lanthanide-encapsulated fullerenes also have potential use as labels that are detected by lanthanide fluorescence. Potential uses include fluorescent labels for tagging of amino acids, antibodies, nucleic acids, and other molecules used in enzymatic analysis, immunoassays, or DNA and protein sequencing applications. The fullerene label is also useful in industrial applications requiring lanthanide labels that can survive extreme chemical environments.

Of all applications proposed for endohedral metallo-fullerenes, their use as radionuclide labels appears to be the most promising. Metallofullerenes are currently quite expensive to produce and purify. Therefore, potential near term applications need to be high value and require only small amounts of material. Radionuclide labels for immunoguided applications fit this requirement exactly. Medical applications are intrinsically high valued, and the amount of material required is extremely small (μg's or less). With future improvements in the areas of metallofullerene production and purification, the cost should be lowered within the next few years. Then the use of a metallofullerene label with a monoclonal antibody would contribute only a small fraction to the total cost of the treatment.

The present experiments demonstrate that endohedral metallofullerenes can be linked to antibodies. The results have significant implications for all applications involving transport of toxic metals in vivo. This research will lead to the development of a new method of transport of toxic metal atoms in vivo, possibly providing an improved method of guiding the radionuclide to the tumor in RTT and RII.

Replacing chelates with metallofullerenes requires a significantly different, but probably advantageous, procedure for the attachment of the radionuclide to the mAb. Many targeting strategies for RIT have been developed. To focus on the correct one requires significant knowledge of the binding strengths, specificity, and pharmacokinetics of the different pieces of the metallofullerene-bispecific antibody being assembled. The available fullerene biodistribution studies, important if multistep targeting approaches are considered, all conclude that fullerenes are nontoxic.

The distributions are dependent on the functionality used to induce water solubility in the fullerene.

A. Metallofullerene Background

Fullerenes are a new class of hollow, closed shell, all carbon molecules discovered by R. E. Smalley and co-workers in 1985 (Kroto et al. 1985). The feasibility of encapsulating metal atoms inside the fullerenes was proven by Smalley's group (Heath et al. 1985) shortly after their initial discovery. Subsequent gas phase photofragmentation experiments showed that fullerenes are among the most stable molecules known to exist., able to store up to 50 eV (delivered by photon bombardment) in internal energy (Wurz and Lykke, 1992). The first bulk amounts of fullerenes were synthesized by resistive heating of graphite in an inert He atmosphere in 1990 by Huffman and Kratschmer (Kratschmer et al. 1990).

Bulk quantities of fullerenes containing La atoms were first synthesized by R. E. Smalley'group in 1991 (Chai et al. 1991). The first metallofullerene samples were produced by laser vaporization of La₂O₃ impregnated graphite rods in a tube furnace at 1200° C. Later experiments demonstrated that endohedral fullerenes could also be produced in usable amounts by the conventional arc evaporation of metal oxide or metal carbide impregnated graphite rods (Johnson et al. 1992). Electron paramagnetic resonance (EPR) studies of toluene soluble La@C₈₂ later proved that the La atom was in the +3 valence state with the fullerene functioning as a compensating anion (Johnson et al. 1992). Recent X-ray diffraction studies on Y@C₈₂ prove conclusively that the metal atoms are encapsulated inside the fullerene (Takata et al. 1995).

It has since been shown that it is possible to produce bulk amounts of fullerenes containing Ca, Sr, Ba, Sc, Y La=lanthanides, and U as well as fullerenes containing multiple metal atoms such as Y dimer and Sc trimer (Bethune, 1993). In general, the production of metallofullerenes creates a broad range of endohedral species containing from 60 to 200 carbon atoms and one or more metal atoms. However, attempts at large-scale encapsulation of metals outside of Group II and Group III have met with little success. High pressures can induce noble gas atoms to enter the cage in very small quanities (Saunders et al. 1996). A recent report of ^{99m}Tc encapsulation (Karem et al., 1997) lacks the definite photofragmentaion test (Heath et al., 1986) to prove encapsulation rather than external complexation.

Metallofullerenes are made using stable isotopes of the lanthanides, and later activated under a slow neutron flux to form radionuclides. By first producing, purifying, and derivatizing the non-radioactive metallofullerene, the time from acquisition of the radionuclide to its administration for RIT is greatly reduced. Thermal neutron activation of ¹⁶⁵Ho@C₈₂ fullerenes has been used to create ¹⁶⁶Ho@C₈₂ fullerenes with up to 8% survivability after 5 hours of irradiation under a flux of 4*1013 neutrons/cm²/s (Cagle et al., 1996).

The low yield appears to be due to the fast neutron component, perhaps 0.25% of the neutrons, although recoil from prompt gamma emission may also play a role in degradation of the fullerene cage. The present experiments are extended to include Ho@C₈₂ fullerenes derivatized to be water soluble. Neutron activation of endohedral metallofullerenes offers a viable path for encapsulation of radionuclides, but further restricts the choice of radionuclide to one that can be readily produced by neutron activation. However, the surviving metallo-fullerenes are ready for immediate use, whereas the slow uptake of short-lived isotopes into chelates reduces their effectiveness.

Despite the limitations imposed by the elements which can be encapsulated and then neutron activated, a wealth of potential radionuclides remain. Table 1 depicts the potential lanthanide beta-emitters with a thermal neutron capture cross sections of about 10 barns. The ¹⁶⁶(Dy/¹⁶⁶Ho pair is particularly intriguing, as it offers the chance to deliver two β⁻ particles per radionuclide.

TABLE 1
Potential lanthanide radioisotopes.

| | Production Cross | | β-energy |
|---|---|---|---|
| Isotope | Section (barns) | half life | (MeV) |
| ¹⁴⁰La | 9 | 40 h | various; to |
| | | | 2.2 |
| ¹⁴²Pr | 12 | 19 h | 2.2 |
| ¹⁶⁶Dy | 1000* | 82 h | 0.5 |
| ¹⁶⁶Ho | 65# | 27 h | 1.8 |
| ¹⁷⁰Tm | 105 | 129 d | 1 |
| ¹⁷⁵Yb | 65 | 4.2 d | 0.5 |
| ¹⁷⁷Lu | 25* | 7 d | 0.5 |

*indicates that two steps are required, but the second step has a cross section of at least 1000 b.
#indicates that it is also produced as the daughter of the ¹⁶⁶Dy decay. All production cross sections are based on neutron capture by the A-1 isotope.

Unlike chelates, fullerene cages withstand recoil energies between 10 and 100 eV (Kikuchi et al., 1994). Early studies suggested that recoil energies less than an electron volt are likely to rupture chelating bonds (Asano et al., 1974; Glenworth and Betts, 1969; Glenworth 1961), although more recent work asserts that all of the ¹⁶⁶Ho formed from ¹⁶⁶Dy decay (recoil energy of 1.85 eV) is retained in the DPTA chelate (Smith et al., 1995). Highly energetic decays with larger recoil energies are desirable because they improve the dose per radioisotpe, as long as the radiated particle pathway is not much larger than the tumor. As blood flow to the center of the tumor is usually severely restricted, more energetic decays offer improved therapy against larger tumors. Losing the metal during recoil (a problem with chelates but not fullerenes) may result in additional toxicity, particularly from the lanthanides which have biological half lives on the order of a decade. This is especially relevant as much recent work has been devoted to the lanthanide beta emitters ⁹⁰Y and ¹⁶⁶Ho, amongst others.

These experiments will concentrate on producing, purifying, and derivatizing Ho@C₈₂. There are three main reasons for concentrating on C₈₂ based compounds. First, for reasons that are not well understood, slightly more C₈₂ lanthanide fullerenes are produced than other metal-containing fullerenes. Second, unlike other lanthanofullerenes C₈₂-based metallofullerenes are relatively stable with respect to oxidation in air and should be chemically similar to empty fullerenes (Suzuki 1993). Recently, the synthesis of derivatized metallofullerenes has confirmed this hypothesis (Suzuki et al. 1995 and Akasaka et al. 1995). Third, only C₈₂ metallofullerenes are soluble in organic solvents such as toluene and can be extracted and purified using currently available chromatography methods.

B. Fullerene Pharmacology

Research into the biological attributes of fullerenes as a class has only just begun. Several preliminary studies indicate that the fullerenes are well tolerated, are relatively nontoxic in vivo, and are certainly much less toxic than free lanthanide ions. The results of these studies are summarized infra.

Because of concerns of the potential carcinogenic effects of benzene and related polycyclic aromatic compounds (of which fullerenes, because of their n-conjugated surfaces might be considered similar), a study on the effect of topical fullerenes was undertaken by Nelson et al. (1993). They demonstrated a lack of carcinogenic effects from C₆₀ and C₇₀ after acute and subchronic applications to mice.

Tours and coworkers (W. A. Scrivens et al. 1994) produced ¹⁴C labelled C₆₀ suspensions (particle size 0.30 μm average diameter) in water and studied its uptake into human keratinocytes. The keratinocytes were exposed to the labelled fullerenes (32,000 dpm, 1.3 μM) and the uptake of the labelled C₆₀ into the cells was monitored. After 6 hours approximately 50% of the applied radioactivity was taken up by the keratinocytes with no further increase noted for longer exposures. Washed cells continued to contain the suspended fullerenes over the next 11 hours. Experiments to determine the effect of the C₆₀ suspension on the proliferation rate of human keratinocytes and fibroblasts by monitoring of labeled thymidine uptake showed fullerenes had no effect on the rate of thymidine uptake.

The only systemic toxicity studies reported in the literature so far are those performed by R. F. Schinazi et al. (1994). In this study, a water soluble fullerene based HIV protease inhibitor was administered to groups of 6 mice at dosages of 15, 30, and 50 mkd. After a slight decline in weight in the treated and control groups (except for the untreated control group) all of the animals gained weight over the period of observation (see FIG. 11) and none of the animals died. There was no statistical difference in the weight between the treated versus control groups and the authors concluded that their fullerene derivative is well tolerated up to a dose of at least 50 mkd. Continued monitoring showed none of the animals had died at 2 months after the start of the experiment.

The in vivo distribution of a particular water soluble ¹⁴C labeled fullerene administered to mice has also been determined (Yamago et al. 0.1995). After injection, the compound moved quickly to the liver and then was distributed to various other tissues. No acute toxicity was noted at doses as high as 500 mg/kg, and all of the mice survived the one week test period. However, excretion of the compound was slow, with 90% being retained after one week. It also appears that the water solubilizing functional group, which contained the ¹⁴C label amid several ester linkages, was metabolized off of the fullerene. This suggests that the distribution and biological half life of fullerene derivatives may depend on the type of functional groups attached to the fullerene. Interestingly, in spite of the fact that it is a fairly large molecule, the fullerene derivative was able to cross the blood brain barrier, a fact that could be very important for future metallofullerene labelled pharmaceuticals.

The biodistribution in mice of hydroxylated lanthanide-C₈₂ encapsulates is under current investigation. The results presented here are regarded as preliminary as the sample is still small. Studies continue under a NIH Phase I grant. The neutron irradiated samples, along with a ¹⁶⁶Ho³⁻ control sample, were used to perform a γ-camera imaging study on four 300 g Sprague-Dawley rats. Two rats were injected intraperitoneally (IP) with 54 and 18 μCi of ¹⁶⁶Ho activity from samples containing irradiated Ho-metallofullerois. Two control studies were performed by 1% administration of approximately 660 μCi of ¹⁶⁶Ho³⁺ in a 1% sodium citrate buffer. The anesthetized rats were scanned side-by-side on a single stage with a γ-camera starting 1 hour after injection and periodically thereafter over the ensuing 48 hours.

The in vivo imaging of the control rats showed rapid ¹⁶⁶Ho³⁻ urinary clearance commencing, as soon as 1 hour post-injection. After 24 hours, essentially all ¹⁶⁶Ho activity had been excreted from the controls. In contrast, the ¹⁶⁶Ho activity from the Ho-metallofullerois produced a discernible image for up to 48 hours and showed an even distribution throughout the blood pool. Essentially no tissue localization was evident. After 48 hours, the ¹⁶⁶Ho activity in the test rats became too low to image effectively (¹⁶⁶Ho t=26.8 hours).

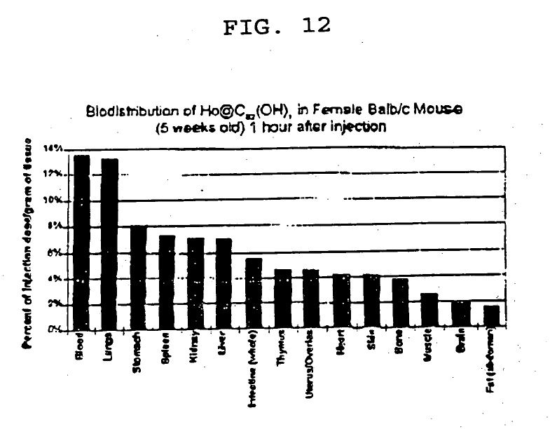

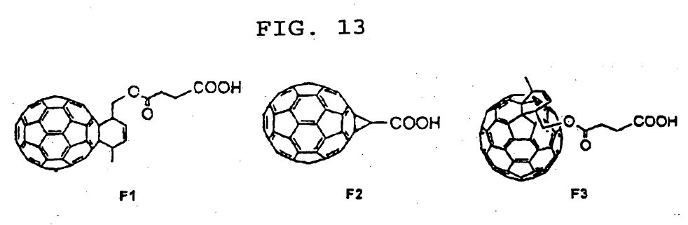

A second biodistribution study was performed on a single. Balb/c mouse to provide a more detailed view of how the Ho-metallofullerol localized in vivo. The results of this study are shown in FIG. 12. Counting of the tissues shown was performed 1 hour after injection with the activities related back to the initial injected dose and corrected for the half-life decay. Additional ¹⁶⁶Ho activity was also counted in the animal's cage, indicating that some clearance of the compound had occurred in the 1 hour before analysis.

Whereas previous fullerene biodistribution studies have demonstrated rapid liver uptake and retention, both of the present studies show that this is not the case for the metallofullerol samples. It appears that the type of derivation used to water-solubilize the fullerene is an important factor in determining its in vivo localization. From a RIT point of view, these initial metallofullerol results are very encouraging. While the long-term biological fate of the presently studied Ho-metallo-fullerol is unclear because of the short ¹⁶⁶HO half-life, it seems rather certain that the observed biodistribution differs from that of simple lanthanide salts as well as underivatized fullerenes. Further testing with samples of higher activity and longer-lived radioisotopes will be necessary to fully resolve this question.

A very recent study reported by Dugan et al. in the 19 Aug. 1997 Proceeding of the National Academy of Sciences reports that water-solubilized fullerenes “act as an effective anti-oxidant”, sweeping up free radicals. Their studies, conducted on oxygen-and glucose-starved nerve cells which build up damaging free radicals, showed that the addition of a water-soluble fullerene derivative (derivatized by carboxylic acid groups) could cut cell death by 75%. Administration of the compound to mice bred to mimic familial amryotrophic lateral sclererosis (Lou Gehrig's disease) delayed the onset of symptoms by 10 days and increased their average 130 day life-span by 9 days. Dosing was accomplished by mini-osmotic pumps starting at 73 days of age and continuing until death. The administered dose corresponded to 15 mg/kg/day, and an equivalent dose for a typical 70 kg human (1.05 g) far more than that required for RIT. There appears to be no toxic effects from doses in this size range. The use of polyhydroxylated fullerols similar to those investigated in this project an non-toxic in vivo free radical scavengers has also been demonstrated (Tsai et al. 1997 and Chueh et al. 1997). From the limited number of studies that have been performed, it seems that the fullerene cage unit is non-toxic. Subject to further, more detailed investigations, it appears the future of fullerenes and metallofullerenes in medicine is quite promising.

C. Three Fullerene Antibodies

Excellent, high-affinity poly-clonal antibodies to fullerenes have been prepared by essentially conventional methods. Anti-fullerene anti-body formation proves that fullerene compounds are processed by the immune system in the same way as any other small molecule antigens. Specificity of the antibodies was confirmed by both binding and inhibition studies.

The anti-fullerene antibodies were produced by immunization of mice with a bovine thyroglobulin (TG) conjugate of a fullerene hemisuccinate F1 containing ca. 10-12 fullerenes per TG molecule (FIG. 13). After a primary immunization in Freund's adjuvant and two subsequent booster injections, the antibody response was confirmed by ELISA. The ELISA plate was coated with F1-rabbit serum albumin (RSA). Preimmune and post-immune sera were examined. Development was with horseradish peroxidase-labeled anti-mouse IgG, using o-phylenediamine as substrate. A high titer of antibody was confirmed (FIG. 4, “First Series of Experiments”)

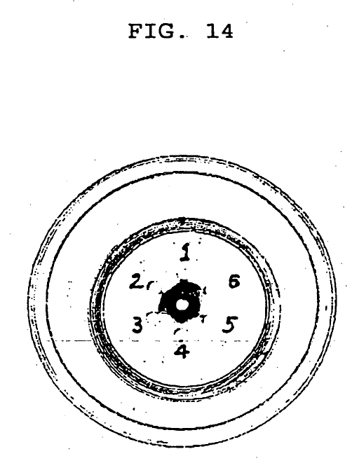

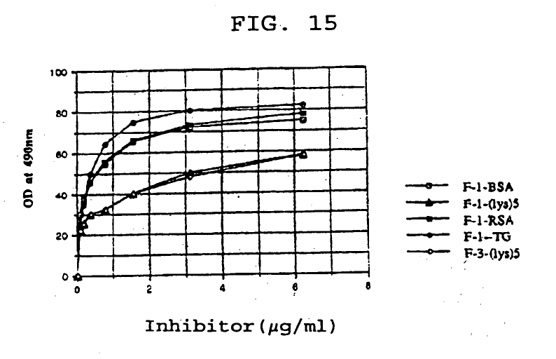

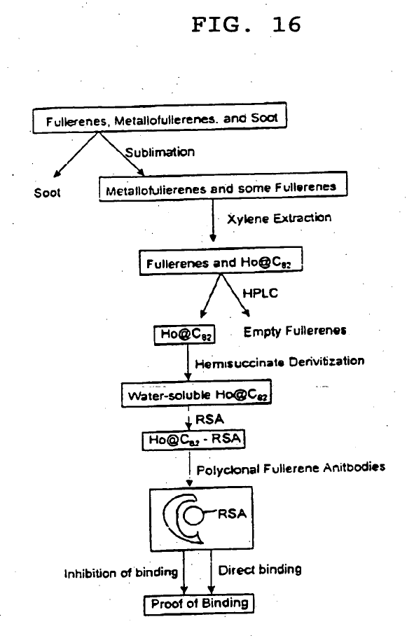

Specificity of the antibodies was confirmed by double diffusion in agar (FIG. 14). Lines of precipitation are seen in wells #1, 2, 3, 4, and 5, with a spur of #2 precipitate “pointing” to F1-BSA. This implies an additional reactive epitope in the F1-TG not present in its BSA conjugate. The overall interpretation of this experiment is that a population of C₆₀-specific antibodies was produced, a subset of which also bound F3, a C₇₀ derivative.

The specificity of the antibodies was confirmed by ELISA inhibition studies (FIG. 15). Binding to F1 covalently linked to RSA was inhibited by F1-BSA, F1-RSA, F1-TG, F3-pentalysine, and F1-pentalysine. The pentalysine derivatives contained ca. 2.8 fullerenes per pentalysine molecule and were water soluble. This demonstrates that the antibody is specific for the fullerenes, not the conjugates. As the polyclonal Abfs also bind C₇₀₁ a fullerene of intermediate size and somewhat more elongated shape, the likelihood of them accommodating the C₈₂ cage seems very high.

Success at binding metallofullerenes to antibodies has profound implications for all techniques involving the transport of metals in vivo. While the fullerene keeps the metal completely contained, the Abf provides a handle for manipulation of the fullerene in vivo.

D. Radioimmunotherapy with Metallofullerenes

This work is aimed at RIT for several reasons. Primarily, the radioactive metals allow the most sensitive measurement techniques to be applied to tracking the development of the process, both in vivo and in vitro. Secondly, RIT has attracted much attention due to its great promise, but requires a technological breakthrough before it can be widely applied. A recent review (Wilder et al., 1996) summarizes the very low response rates in clinical trials of RIT. Recent research has been towards shorter half-life radioisotopes, metabolizable linkages, and rapid clearing of the chelated radioisotope. These strategies are attempts at reducing the background dosage when the mAb does not rapidly find the tumor, compensating for its eventual separation from the radioisotope. If all metal atoms remain encapsulated over any length of time, as is true for metallofullerenes, the radionuclide is never separated from the mAb by leaving the chelate. Longer-lived radionuclides, such as ¹⁷⁷Lu, suffer fewer decays while the mAb is locating the tumor, and therefore take best advantage of the metallofullerene delivery for one-step targeting (Schlom et al., 1991).

However, a single dosage of the fullerene-bispecific antibody may not be the best way to apply fullerenes to RIT. Some of the conventional problems have been mitigated by two-(Hnatowich et al., 1987) and three-step (Paganelli et al., 1991) targeting approaches. Use of metaollothone in (−7 kDa) as a chelator is attractive because it is readily fused to other proteins by recombinant DNA technologies (Virzi, et al., 1995). However, its chelation ability was very poor in comparison to the more recent advanced synthetic chelating agents. A more common approach has been to link a good chelator (loss of −1% of metal atoms per day) to biotin (Vitamin H). Such assemblies have slightly lower molecular weights than fullerenes, indicating rapid circulation (and clearance) in vivo. Some problems have been encountered with cleavage of the chelator from biotin (Rosebrough, 1993). In the three step program (Paganelli et al., 1991), a bispecific mAb, combining biotin with the antigen-specific component, is administered first. Avidin, which has an association constant for biotin of order 1015, is added in the second step. In the final step, the biotin-chelate conjugate binds to the avidin. Even with this optimized administration program, only 0.012% of the injected dose was delivered to the tumor.

Similar multi-step approaches may be suitable for metallofullerene derivatives, depending on their pharmacokinetics. One possible strategy is to first administer a bispecific antibody that consists of an antigen-specific component and the Abf, followed by the metallofullerene. The available biodistribution studies of fullerenes indicate that, without stable functionalization to induce water solubility, they rapidly accumulate in the liver, from which clearance requires days. However, when water solubility is maintained, the mouse biodistribution indicates that fullerenes do not accumulate in any particular organ. As there are no natural receptors for fullerenes, they may exhibit very high in vivo selectivity for the Abf.

II. Experimental Design and Methods

The goal of the Phase I project is to demonstrate that endohedral metallofullerenes can be attached to fullerene antibodies. While these experiments will isolate monoclonal antibodies for C₈₂ with endohedral metal atoms, the work begins with the polyclonal mixture bioengineered in response to empty C₆₀. Four tasks consistent with this specific aim have been formulated: l) Synthesize holmium containing fullerenes; 2) Purify the selected Holmiom fullerene; 3) Derivatize the purified Ho@C₈₂ to make it water soluble; 4) Bind the derivatized Ho@C₈₂ to the fullerene antibodies. FIG. 16 shows the flow of the research studies.

A. Production of Holmium Metallofullerenes

The evaporation of graphite using a carbon arc in an inert atmosphere was the first successful approach for the production of macroscopic amounts of fullerene materials. This technique has also proven to be the method of choice for the production of macroscopic amounts of endohedral metallofullerenes as well. The graphite that is vaporized is doped with the desired metal in a form, such as Ln₂O₃, that is readily reduced to the neutral metal during vaporization. The metallo-fullerenes utilized in this project are likely to be produced by the conventional carbon arc method as described below.

A current Phase II project is underway to produce bulk (kg) quantities of fullerenes and metallofullerenes using a continuous, combustion based process that can employ organometallic precursors. This process will substantially lower the cost of all types of fullerenes. Since its success is not guaranteed, described herein is a proven method for production of fullerenes and metallofullerenes.

A unique carbon arc reactor capable of both producing fullerenes and separating all the fullerenes from the raw soot has been designed. Because some fullerenes are radicals, all processing is performed under anaerobic conditions. The reactor is constructed primarily from quartz and serves to both vaporize the carbon rods and to perform an initial sublimation step. It is capable of generating several hundred milligrams of sublimed fullerene/metallofullerene mixture per 5″ length of ¼″ rod vaporized. The reactor is currently run in a single rod made by butting the ¼″ rod to be vaporized against a larger ½″ diameter rod. The arc is run in DC mode with the polarity being reversed every few minutes to insure uniform evaporation. Multiple rods can be sequentially vaporized without venting the system by loading new rods into the reactor through the ball valve. Metal oxide doped carbon rods are produced by incipient wetness impregnation, a procedure developed at TDA and adequately described in the literature (Cagle et al., 1996). During the vaporization step, the fullerene collector is replaced by a seal to keep soot from going past the quartz baffle plate, and the tube furnace is removed to allow dissipation of the excess heat. Analysis of soot samples from different areas of the reactor after vaporizing one undoped graphite rod showed the yield of empty fullerenes (determined by quantitative HPLC) to be 12%.

After evaporating several sets of graphite rods, the apparatus is pumped out to a vacuum of −10 mtorr, and the electrodes are withdrawn. The furnace is replaced, and the quartz vessel is heated to 250. ° C. to allow any volatile impurities to be pumped away. The water cooled collector is now inserted through the gate valve, and the furnace is programmed to heat to 750° C. over several hours. The collector provides a water cooled surface area of 65 cm² upon which the fullerenes and metallofullerenes condense. Typical films, depending on the quantity of evaporated material, are on the order of 20-100 μm thick. Depending upon the metal, approximately 200-1000 mg of sublimed material can be collected. From our current data, it is estimated that the yields collected by sublimation are equivalent to that collected by solvent extraction, but in contrast to solvent extraction, all of the small gap empty fullerenes and endohedral metallofullerenes produced (up to about 100 carbon atoms) readily sublime onto the collector. By performing the sublimation in two steps, at least 30% of the C₆₀ and C₇₀ can be removed in a first pass at 500 C, without losing significant amounts of Ln@C₈₂. This renders the higher temperature sublimate more concentrated in Ho@C₈₂ crucial to its efficient purification (vida infra).

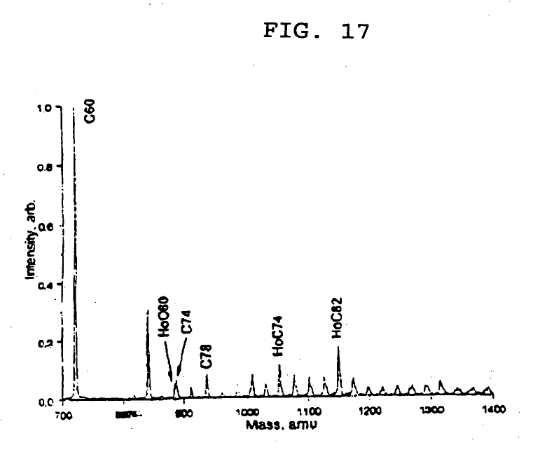

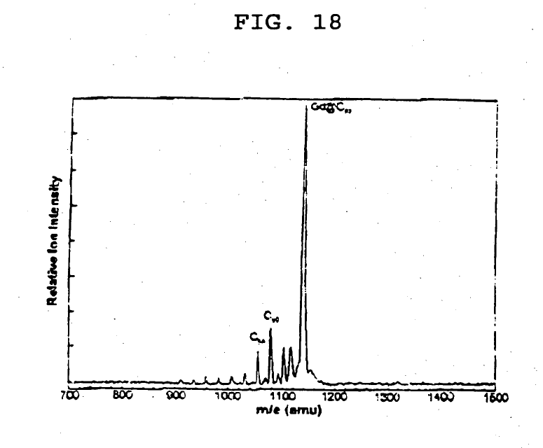

A mass spectrum (MS) of a typical sublimed sample (single step at high temperature) of Ho containing fullerenes is shown FIG. 17. This spectrum was obtained with TDA's laser desorption time-of-flight reflectron mass spectrometer. Metallofullerene samples were deposited on a stainless steel target disk from suspensions made by sonicating the metallofullerene solid in ethanol.

Desorption and ionization were performed with 355 nm light from a Q-switched Nd-YAG laser. Peak intensities between the empty and metallofullerenes are not always quantitative, since metallofullerenes are easier to ionize than the empty fullerenes. In this case, empty fullerenes require three 355 nm photons to ionize while metallofullerenes require only two. The net result is an apparent enhancement of the metallofullerene signal (we estimate by a factor of two, based on measurements made using 157 nm light where 1 photon ionizes all of the fullerenes.) This makes Ho@C₈₂ about as abundant as the higher empty fullerenes such as C₈₄.

B. Purification of the Ho@C₈₂

Fullerenes from the concentrated sublimate is dissolved in xylene. Among lanthanide endohedral metallofullerenes, only the C₈₂ encapsule is soluble, which provides a rapid means of achieving purity among endohedral fullerene cage sizes. After filtering to remove the insolubles, the extract is separated into its components using a semi-preparative high pressure liquid chromatography (HPLC) technique. Commercial columns with stationary phases have been developed specifically for separating fullerenes. A variety of these fullerene HPLC columns are available and it has been found that the Cosmosll PYE (2-(1-pyrenyl)ethyl) column is the best for Ln@C₈₂ separations. Much expertise in HPLC separation of Ln@C₈₂ has been acquired as the result of a previous study of Gd@C₆₂ as an MRI contrast agent. Thus, examples are cited from that procedure. The procedure for optimizing the collection of Ln@C₈₂ was developed during that work. It is not expected that the change from Gd to Ho will result in any significant behavioral differences in the HPLC of the endohedral metallofullerenes.

While previous work reported in the literature performed HPLC under aerobic conditions, (see for example Funasaka et al. 1995) these studies determined that anaerobic controls better preserved the fraction of Gd@C₈₂ in solution. (The hydroxylated, water soluble Gd@C₈₂(OH)ₓ derivative does not appear to be air sensitive.) Pure, degassed o-xylene was used as the mobile phase at a flow rate of 8 ml/min, and all handling of the sample solutions was performed using anaerobic Schlenk line techniques. The fraction containing the Gd@C₈₂ was identified using MS. The solubility of fullerenes in o-xylene is about three times that of toluene and this greatly increases the ratio of fullerenes in solution to those retained on the stationary phase. Therefore, the capacity or amount of fullerenes purified per injection was very large. Each injection contained 2 ml of saturated fullerene o-xylene solution (about 30 mg of fullerenes).

The Gd@C₈₂ fraction was collected under Ar as it eluted from the column. The final solution, shown in FIG. 18, was then stored under Ar until ready for derivatization. Because we started with a highly enriched fraction and handled it anaerobically, the purity of the samples is quite high (the integrated area is −95% Gd@C₈₂, but as explained earlier, the absolute calibration is uncertain). The need for only a single, short HPLC step greatly facilitated separation of large quantities of Gd@C₈₂, and the production and purification process was then repeated to successfully produce approximately 25 mg of the material shown in FIG. 11. Repeating this procedure for holmium fullerenes will collect an appropriate amount to prepare the water soluble derivative.

C. Producing the Water Soluble Holmium Fullerenes

The Ho@C₈₂ will need to become water soluble in order to attach it to the fullerene antibodies. A wide variety of reactions have been shown to make C₆₋₀ water soluble. Functionalization of the outside of the cage with about thirty hydroxyl groups has also been shown to be effective for endohedral metallofullerenes. One or more carboxylic acid groups have been linked to the fullerene in a variety of ways to induce water solubility. Inclusion in cyclodextrin and poly(vinylpyrrolidine) have also been successful. Polyhydroxylation, cyclodextrin inclusion, and complexation with poly(vinylpyrrolidine) are expected to alter the exterior of the cage beyond the recognition of the Abfs. Therefore, a carboxylic acid group is linked to the metallofullerene.

While only one other reaction of Ln@C₈₂ has been demonstrated (besides polyhydroxylation, and that other one does not produce a water soluble derivative), the chemistry of the endohedral metallofullerenes is likely to be very similar to that of the empty fullerenes. The organic chemistry of empty fullerenes is a rapidly growing field, led by reactions of C₆₀. All chemical principles demonstrated on C₆₀ also hold for larger fullerenes, although the isomeric mix of products varies according to the cage size and shape. The hemisuccimide derivative used in the initial preparation of C₆₀ antibodies was also demonstrated for C₇₀, and the same procedure will be followed for the Ho@C₈₂.

To prepare the empty fullerene derivatives (Lu, 1995), trans-trans-2,4 hexadien-lol was added to C₆₀ in toluene under inert atmosphere. The mixture was then heated to 80° C. overnight. After cooling, the solvent was removed. The residue was purified by flash column chromatography with toluene, followed by 20:1 toluene:ethyl acetate.

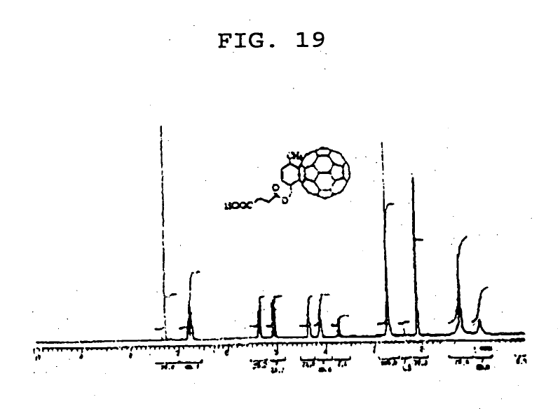

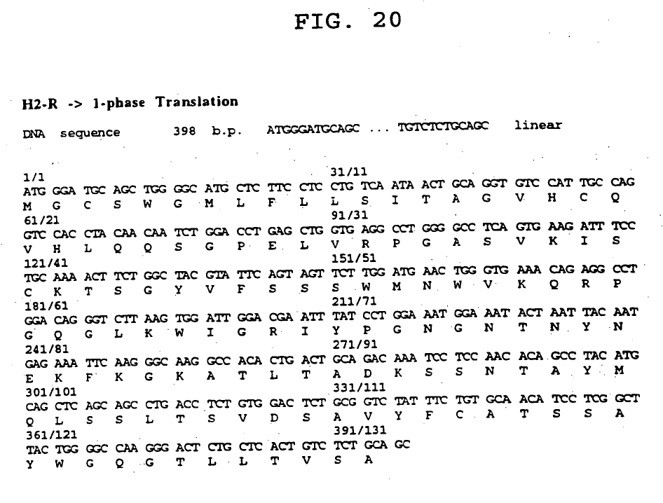

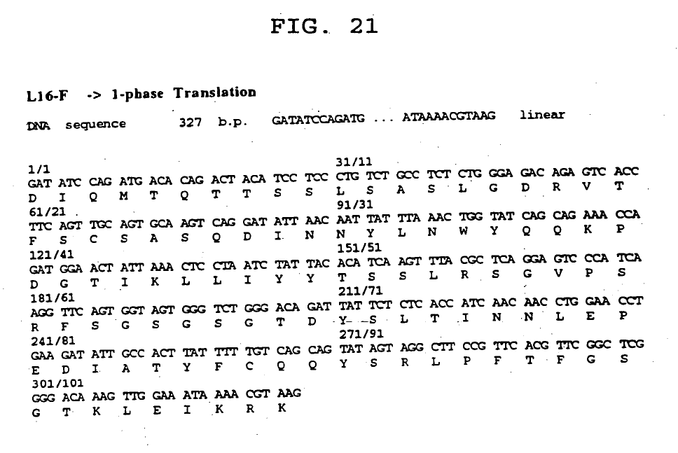

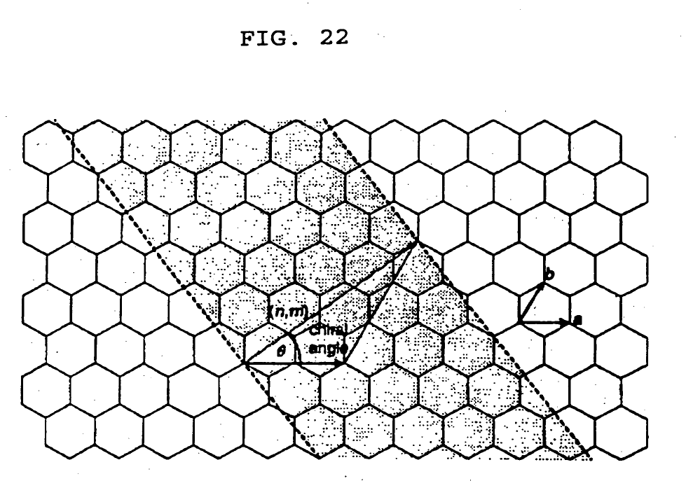

That product was treated with dimethylaminopyridine and succinic anhydride under inert atmosphere in toluene:methylene chloride 1:1 solution. This mixture was warmed to 55° C. for one day, followed by cooling and solvent removal. The residue was purified by flash column chromatography with methylene chloride followed by methylene chloride:ethanol>15:1. The product, a dark brown solid, is shown in FIG. 19. These same steps are followed for Ho@C₈₂.

While the procedure above creates a fullerene that remains water soluble only as long as the ester linkage remains intact, the F2 structure (see FIG. 13) results in a fullerene that is likely to remain water soluble under more adverse conditions. Its preparation is also a two step synthesis (Issacs and Diederich, 1993). This fullerene, or another ester-less derivative, may be used when maintenance of the solubility of fullerenes not bound to the Abf is desirable.

D. Binding the Derivatized Ho@C₈₂ to the Fullerene Antibodies

As for the preparation of the hemisuccinate derivative, the procedure for binding the Ho@C₈₂ derivative to the Abf follow the procedure established for the empty fullerenes. The procedure for linking the Ho@C₈₂ hemisuccinate to the RSA and pentalysine proceeds via reaction with N-hyroxysuccinimide and dicyclohexylcarbodiimide in pyridine. After two days at room temperature, crystals of dicyclohexylurea (a by-product of the reaction) needs to be removed by centrifugation, leaving a solution of the fullerene N-hydroxysuccinimide ester (NHS). Half of the solution is allowed to react with RSA at a ratio of 15:1 Ho@C₈₂—NHS:RSA. The other half is allowed to react with pentalysine at a ratio of 5:1. This results in 10-12 metallofullerenes per molecule of RSA and 2.5 to 3 metallofullerenes per pentalysine.

Similarly, studies of its binding, and inhibition of its binding are carried out in precisely the same manner described in Section I.C. above. In the direct binding study, the ELISA plate is coated with Ho@C₈₂—RSA. In analogy to the previous experiments, also tested is inhibition of binding of antibody to C₆₀—RSA by Ho@C₈₂—RSA and Ho@C₈₂-(lys)₅. These tests confirm the binding of a metallofullerene to a fullerene antibody.

### REFERENCES FOR FOURTH SERIES OF EXPERIMENTS

1. Arano, Y., K. Wakisaka, Y. Ohmono et al. (1996). “Assessment of radiochemical design of antibodies using an ester bond as the metabolizable linkage: evaluation of maleimidoethyl 3-(Tri-n-butylstannyl)hippurate as a radioination reagent of antibodies for diagnostic and therapeutic applications,” Biconjugate Chem. 7, 628-637.
2. Bethune, D. S., R. D. Johnson, J. R. Salem, M. S de Vries and C. S. Yannoni (1993). “Atoms in carbon Cages: the Structure and Properties of Endohedral Fullerenes,” Nature 366, 123.
3. Buchanan, M. V., and R. L. Hettich (1993). Anal. Chem. 65, 245A.
4. Chai, Y., T. Guo, C. Jin, R. E. Haufler, L. P. F. Chibante, J. Fure, L. Wang, J. M. Alford, and R. E. Smalley (1991). “Fullerenes with Metals Inside,” J. Phys. Chem. 95, 7564.
5. Chiang, L. Y., J. W. Swirczewski, C. S. Hsu, S. K. Chowdhury, S. Cameron, and K. Creegan (1992). “Multi-hydroxy Additions onto C₈₀ Fullerene Molecules,” J. Chem. Soc. Chem. Commun., 1791.
6. Coloma, M. J. and S. L. Morrison (1997). “Design and production of novel tetravalent bispecific antibodies,” Nat. Biotechnol. 15, 125-126.
7. DeNardo, S., P. Gumerlock, M. Winthrop, P. Mack, S. Chi, K. Lamborn et al. (1995). “Yttrium-9 0 Chimeric L6 Therapy of Human Breast Cancer in Nude Mice and Apoptosis-related Messenger RNA Expression,” Cancer Research 55, 125-126.
8. Dugan, L., D. Turetsky, C. Du, et al. (1997). “Carboxyfullerenes as neuroprotective agents,” Proc. Natl. Acad. Sci. USA 94, 9434-9439.
9. Erlanger, B. F. (1980). “Preparation of Antigenic Hapten-Carrier Conjugates,” Methods in Enzymology 70(85).
10. Friedman, S. H., D. L. DeCamp, R. P. Sijbesma, G. Srdanov, F. Wudl, and G. L. Kenyon (˜1993). “Inhibition of the HIV-1 Protease by Fullerene Derivatives: Model Building Studies and Experimental Verification,” J. Am. Chem. Soc. 115, 6506.
11. Gansow, 0. A., M. W. Brechbiel, S. Mirzadeh, D. Colcher, and M. Roselli (1990). “Chelates and Antibodies: Current Methods and New Directions,” in Chancer Imaging with Radiolabeled Antibodies, D. M. Goldenberg, editor, Kluwer Academic Publishers, P. 153.
12. Gecker, K. E. and A. Hirsh (1993). “Polymer-Bound C₆₀” J. Am. Chem. Soc. 115, 3850.
13. Glentworth, P., Betts (1961). “The effect of beta decay on the exchange properties of the rare earth-EDTA complex ions,” Can. J. Chem. 39, 1049.
14. Glentworth, P., C. Wright (1969). “Chemical effects of nuclear transformations of complexed metal atoms-I. The beta-decay of Ce-144 and Ce-143 complexed with polyaminocarboxylic acid chelating ligands,” J. Inorg. Nucl. Chem. 31, 1263.
15. Gromov, A., W. Kratschmer, N. Krawez et al (1997). “Extraction and HPLC purification of Li@C_{60/70},” Chem. Commun., 2003-4.
16. Haufler, R. E. (1994). “Techniques of Fullerene Production,” in Fullerenes: Recent advances in the Chemistry and Physics of Fullerenes and Related Materials, K. D. Kadish and R. S. Ruoff, Editors, p. 50, The Electrochemical Society Press, Pennington, N.J.
17. Heath, J. R., S. C. O'Brien, Q. Zhang, Y. Liu, R. F. Curl, H. W. Kroto, F. K. Tittel, and R. E. Smalley (1985) “Lanthanum complexes of Spheroidal Carbon Shells”, J. Am. Chem. Soc. 107, 7779.
18. Hnatowich, D. J., F. Virzi, M. Rusckowski (1987). “Investigations of Avidin and Biotin for Imaging Applications,” J. Nucl. Med. 28, 12294-1302222.
19. Isaacs, L. And F. Diederich (1993). “Structures and Chemistry of Methanofullerenes: A Versatile Route into N-[(Methanofullerene)caronyl]-Substituted Amino Acids”, Helv. Chem. Acta. 76, 2454.
20. Junghans, R. P., d. Dobbs, M. W. Brechbiel, S. Mirzadeh, A. A. Raubitschek, 0. A. Gansow, and T. A. Waldmann (1993). “Pharmokinetics and Bioactivity of 1, 4, 7, 10-tera-azacyclododecane N, N^{I}, N^{II}, N^{III}-tetraacetic acid (DOTA)-Bismuth-conjugated Anti-Tac Antibody for a-Emitter (212Bi) Therapy,” Cancer Res. 53, 5683
21. Karam, L. R., M. Mitch, B. Coursey (1997). “Encapsulation of ^{99m}Tc within Fullerenes: a Novel Radionuclide Carrier,” App . . . Radiat. Isot. 48 (16), 771-776.
22. Kikuchi, K., K. Kobayashi, K. Sueki, S. Suzuki, H. Nakahara, and Y. Achiba (1994). “Encapsulation of Radioactive ¹⁵⁰Gd and ¹⁶¹Tb Atoms in Fullerene Cages,” J. Am. Chem. Soc. 116, 9775.
23. Klein, K., T. Nguyen, P. Laroque et al. (1989). “Yttrium-90 and Iodine-131 Radioimmunoglobulin Therapy of an Experimental Human Hepotoma,” Cancer Research 49, 6383-6389.
24. Kratschmer, W., L. D. Lamb, K. Fostiropoulos, and D. R. Huffman (1990). “Solid C₆₀: A New Form of Carbon”, Nature 347, 354.
25. Kroto, H. W., J. R. Heath, S. C. O'Brien, R. F. Curl, and R. E. Smalley (1985). “C₈₀Buckminsterfullerene”, Nature 318, 162
26. Le Doussai, J. M., M. Martin, E. Gautherot, M. Delaage, J. Barbet (1989). “In vitro and in vivo targeting of radiolabeled monovalent and divalent haptens with dual specificity monoclonal antibody conugates: enhanced divalent hapten affinity for cell-bound antibody conjugate,: J Nucl. Med. 30, 1358-1366.
27. Leu, J. G., Chen B-X., Diamanduras, A. W., Erlanger, B. Proc. (1994). “Idiotypic mimcry and the assembly of a supramolecular structure; an antidiopic antibody that mimics taxol in its tububulin-microtubulin interactions,” Natl. Acad. Sci USA 91, 10690.
28. Li, M. C. Meares, Q. Salako et al. (1995). “Prelabeling of chimeric Monoclonal Antibody L6 with 90 Yttrium- and 111 Indium-1, 4, 7, 10-tetraazacyclododecane-N, NI, NII, NIII-tetraacetic acid (DOTA) Chelates for Radioimmuno-diagnosis and Therapy,” Cancer Research 55, 5726-5728.
29. Lu, Q. Y. (1995). PhD Thesis, New York University.
30. Meares, C. F. (1986). “Chelating Agents for the Binding of Metal Ions to Antibodies,” Nucl. Med. Bio. 13, 311.
31. Paganelli, G. P. Manani, F. Zito, E. Villa, F. Sudati et al. (1991). “Three-step monoclonal antibody tumor targeting in carcinoembryonic antigenpositive patients,” Cancer Research 51, 5960-5966.
32. Pluckthun, A., P. Pack (1997). “New protein engineering approaches to multivalent and bispecific antibody fragments,” Immunotechnology 3, 83-105.
33. Renn, O., D. Goodwin, M. Studer, J. Moran, V.

Jacques, C. Meares (1996). “New approaches to delivering metal-labeled antibodies to tumors: Synthesis and character-ization of new ciotinyl chelate conjugates for pre-targeted diagnosis and therapy,” J. Of Controlled Release 39, 239-249.
34. Rosebrough, S. F. (1993). “Plasma stability and pharmacokinetics of radiolabeled deferoxaminebiotin dreivatives,” J. Pharmcol. Exp. Ther. 265, 408-415.
35. Rouvier, E., E. Gautherot, P. Meyer, and J. Barbet (1997). “Targeting medullary thyroid carcinomas with bispecific antibodies and bivalent haptens. Results and clinical perspectives,” Hormone Research 47, 163-167.
36. Saunders, M., R. Cross, H. Jimenez-Vazquez, R. Simshi, A. Khong (1996). “Noble Gas Atoms Inside Fullerenes,” Science 271, 1693-1697.
37. Scheinberg, D. A., and M. Strand (1982). “Leukemia cell targeting and therapy by monoclonal antibody targeting in erythroleukemic mice,” Cancer Res. 42, 44.
38. Schinazi, R. F., R. Sijbesma, G. Srdanov, D. L. Hill, and F. Wudl (1993). “Synthesis and Virucidal Activity of a Water-Soluble. Configurationally Stable, Derivatized C₆₀ Fullerene”, Antimicrobial Agents and Chemotherapy 37, 1707.
39. Schinazi, R. F., A. McMillan, A. S. Juodawlkis, J. Phar, R. Sijbesma, G. Srdanov, J. C. Hummelen, F. D. Boudinot, C. L. Hill, and F. Wudl (1994). “Anti-Human Immunodefiency Virus, Toxicity in Cell Culture, and Tolerance in Mammals of a Water-Soluble Fullerene”, in Recent Advances in the Chemistry and Physics of Fullerenes and Related Materials, ed., K. M. Kadish and R. S. Ruoff, The Electrochemical Society Proceedings v. 94-24, (The Electro-chemical Society, Inc. NJ.).
40. Schlom, J., K. Siler, D. Milenic, S. Eggensperger, et al. (1991). “Monoclonal Antibody-based Therapy of a Human Tumor Xenograft with a ¹⁷⁷Lutetium-labeled Immunoconjugate,” Cancer Research 51, 2889-2896.
41. Sijbesma, R., G. Srdanov, F. Wudl, J. A. Castoro, C. Wilkings, S. H. Firedman, D. L. Decamp, and G. L. Kenyon (1993). “Synthesis of a Fullerene Derivative for the Inhibition of HIV Enzymes,” J. Am. Chem. Soc. 115, 6510.
42. Sykes, T., V. Somayaji, S. Bier et al. (1997). “Radiolabeling of Monoclonal Antibody B43.13 with Rhenium-188 for Immunoradiotherapy,” Appl. Tadiat. Isot. 48(7), 899-906.
43. Takata, M., B. Umeda, E. Nishibori, M. Sakata, Y.

Saito, M. Ohno, and H. Shinghara (1995). “Confirmation by x-ray diffraction of the endohedral nature of the metallofullerene Y@C₈₂”, Nature 377, 46.
44. Taylor, R. and D. R. M. Walton (1993). “The Chemistry of Fullerenes,” Nature 363, 685.
45. Virzi, F., P. Winnard, Jr., M. Fogarasi, T. Sano et al. (1995). “Recombinant Metallothionein-Conjugated Streptavidin Labeled with ¹⁸⁸Re and ^{99m}Tc,” Biconjugate Chem. 6, 139-144.
46. Vuillez, J. Ph., D. Moro et al. (1997). “Two-step immunoscintigraphy for non-small-cell lung cancer staging using a bispecific anti-CEA/anti-indium-DPTA antibody and an indium-111-labeled DPTA dimer.,” J. Nucl. Med. 38, 507-511.
47. Wilder, R., G. DeNardo, S. DeNardo (1996). “Radioimmunotherapy: Recent results and future directions,” J. Of Clinical Oncology 144(4), 1383-1400.
48. Wurz, P. and K. R. Lykke (1992). “Multiphoton Excitation, Ionization, and Dissociation of C₆₀,” J Phys. Chem. 96, 10129.
49. Yamakoshi, Y., T. Yagami, K. Fukuhara, S. Sueyoshi, N. Miyata (1994). “Solubilization of Fullerenes into Water with Polyvinylpyrrolidone Applicable to Biological Tests,” J. Chem. Soc., Chem. Commun., 517-518.
50. Yuanfang, Liu, Wu Chuanchu (1991). “Radiolabeling of monoclonal antibodies with metal chelates,” Pure and Appl. Chem. 63 (3), 427-463.
   Fifth Series of Experiments

Various applications are being sought for fullerene-based compounds in fields as diverse as electronics and pharmacotherapeutics. With respect to the latter, it is of interest to determine whether it was possible to produce monoclonal anti-fullerene antibodies. We succeeded in doing so and characterized one of them with respect to its specificity. Its structure and interaction with fullerenes was also ascertained by X-ray crystallography. The same monoclonal antibody cross-reacted with single wall nanotubes as determined immunochemically and by atomic force microscopy. The antibody, therefore, serves as a bridge between two disciplines: biology and electronics.

Single wall carbon nanotubes (SWNTs) (1) are a remarkable new class of nanometer diameter metallic and semiconducting wires that carry current as n electrons propagating on their graphitic surface. They are physically robust, exhibit great tensile strength, do not oxidize or have surface states under ambient conditions, and show high conductivity (2). They are easily grown in lengths of tens of microns and can be precisely positioned and manipulated when attached to AFM tips (3). Their remarkable electrical properties suggest they might be components of some future nanoscale electronics. We report observation of specific binding of a biomolecule to SWNTs.

To preserve electrical conductivity in biologically derivatized SWNT wires, the sp2 graphitic sidewall structure should be minimally perturbed to a specific yet noncovalent contact. A recent inventive approach to this problem involved adsorption of the pyrene moiety of the aqueous bifunctional small molecule: 1-pyrenebotanoic acid succinimidyl ester. Subsequently, the surface-immobilized esters were reacted with several proteins rich in surface amines (4). Previous experiments on multiwall tubes have demonstrated physical adsorption of metallothionein and streptavidin proteins (5, 6).

We now show that a monoclonal antibody specific for C₆₀ fullerenes (7, 8) recognizes and binds specifically to SWNTs. The sequences of the light and heavy chains of this IgG antibody were determined recently, and using X-ray crystallography of its Fab fragment, it was found that the binding cavity was formed by clustering of hydrophobic amino acids (8). An induced fit mechanism participated in the binding of fullerenes, thus suggesting that SWNTs might also be recoginzed.

The immunochemical reaction between SWNTs and the C₆₀ antibody was first demonstrated in an ELISA (9), a procedure in which a known ligand of anti-fullerene and a presumptive one compete for binding to an anti-fullerene antibody that is adsorbed to the surface of a plastic well. The presumptive ligand, in this case, was a colloidal suspension of SWNT. Nonspecific binding was eliminated by the presence of a detergent (0.05% Tween 20). Competition was seen at very high dilutions of the SWNT colloidal suspension. A quantitative measure of binding coefficient will require detailed study as a function of available SWNT surface area, structural type, and extent of SWNT aggregation into ropes.

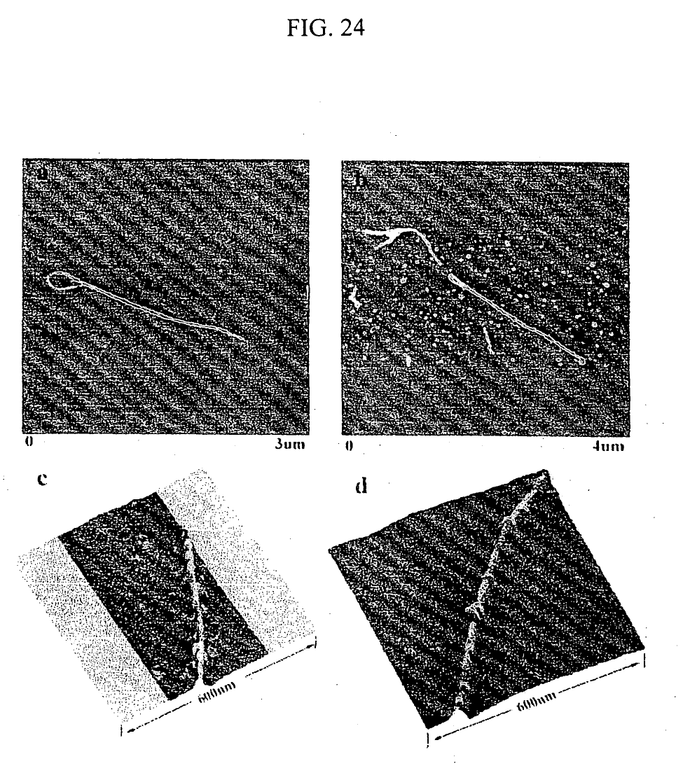

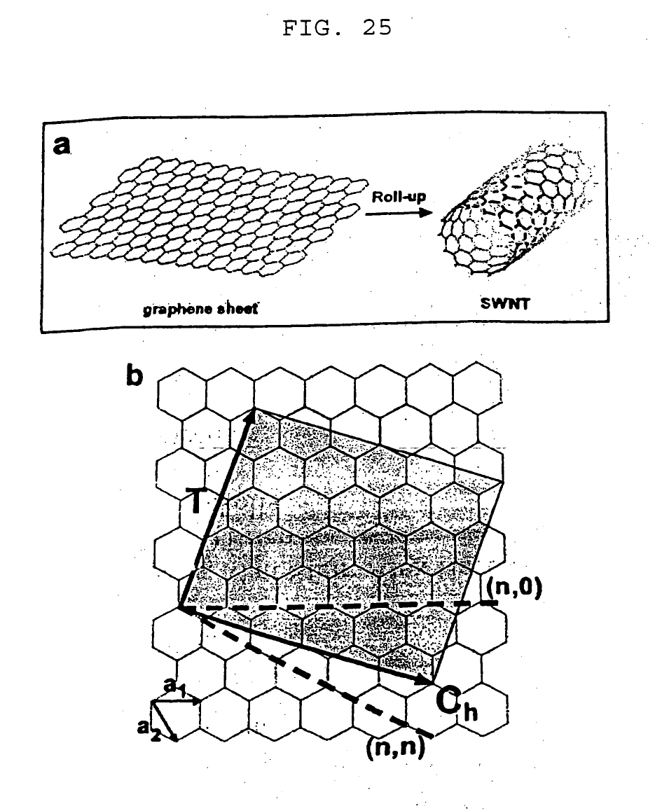

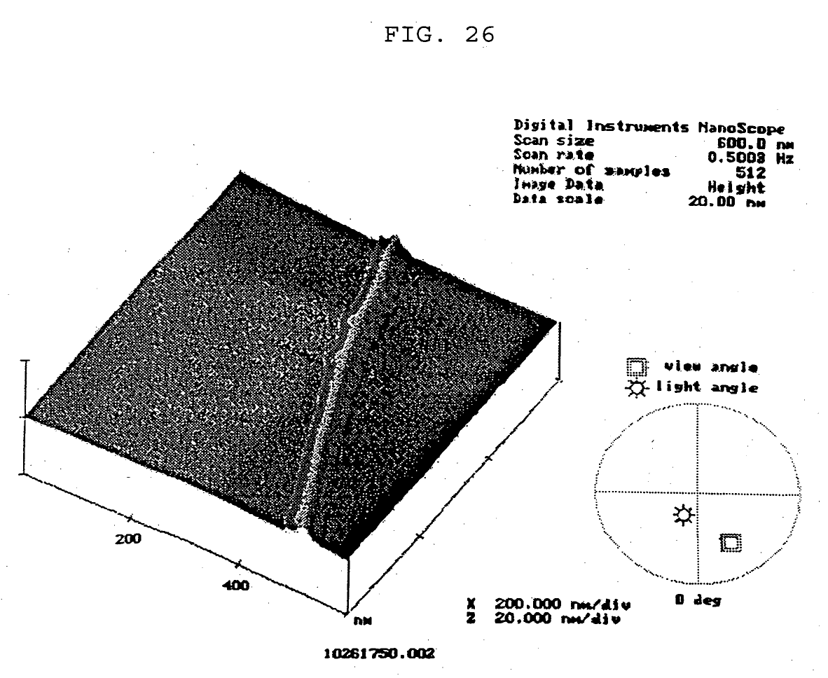

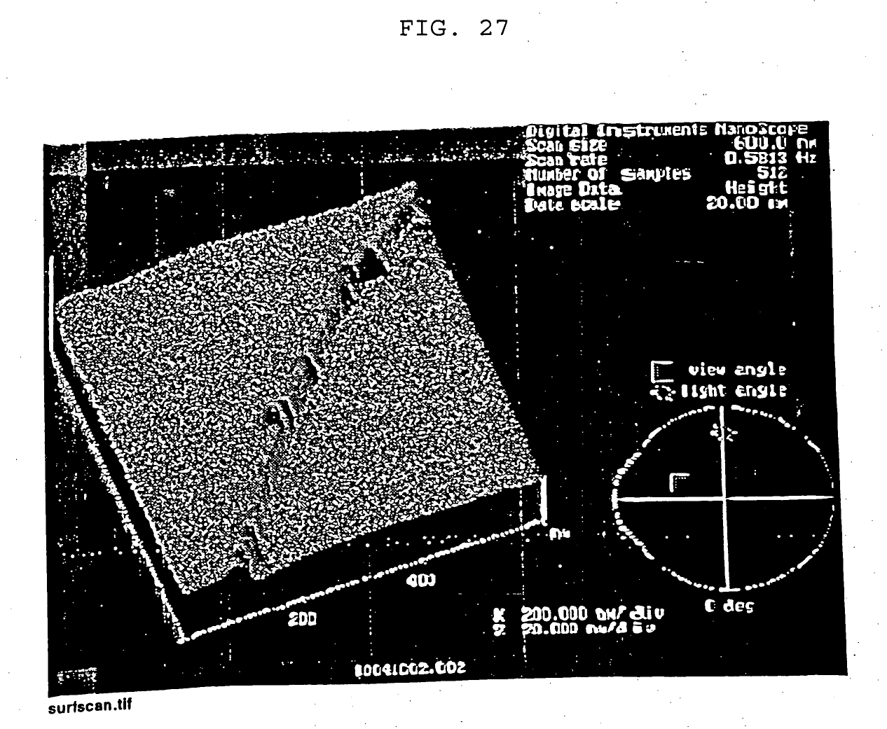

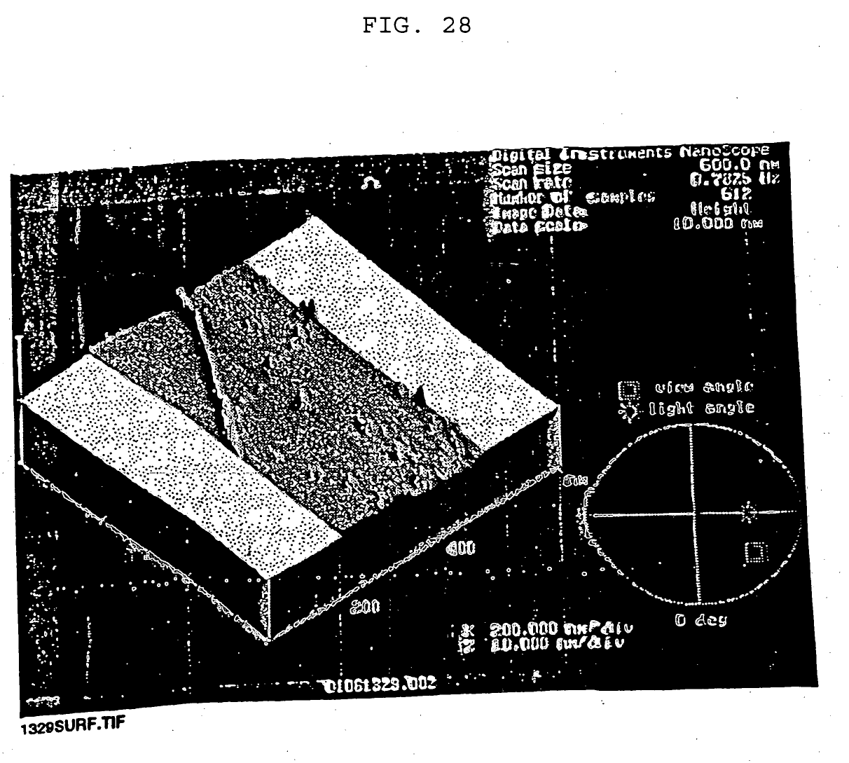

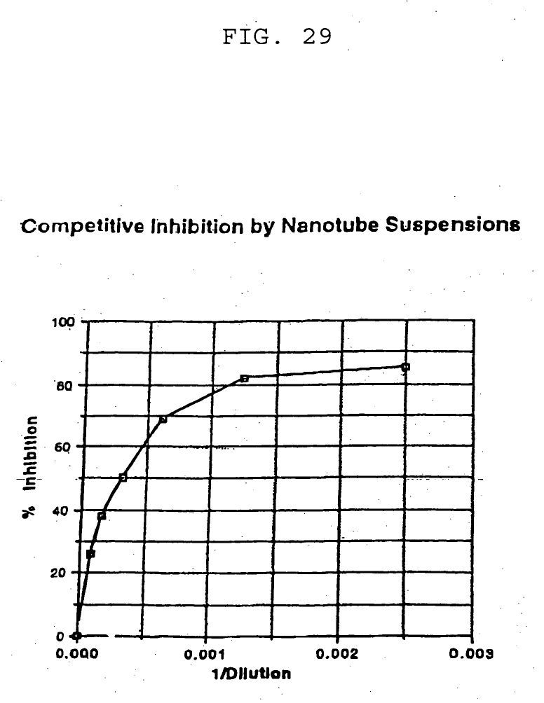

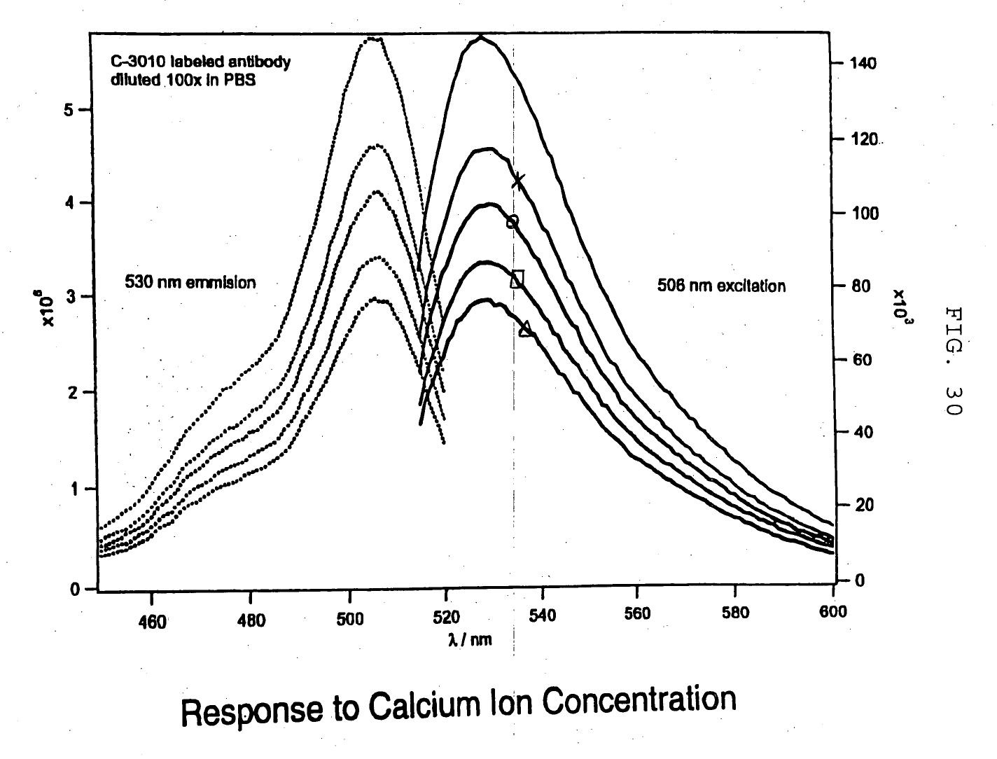

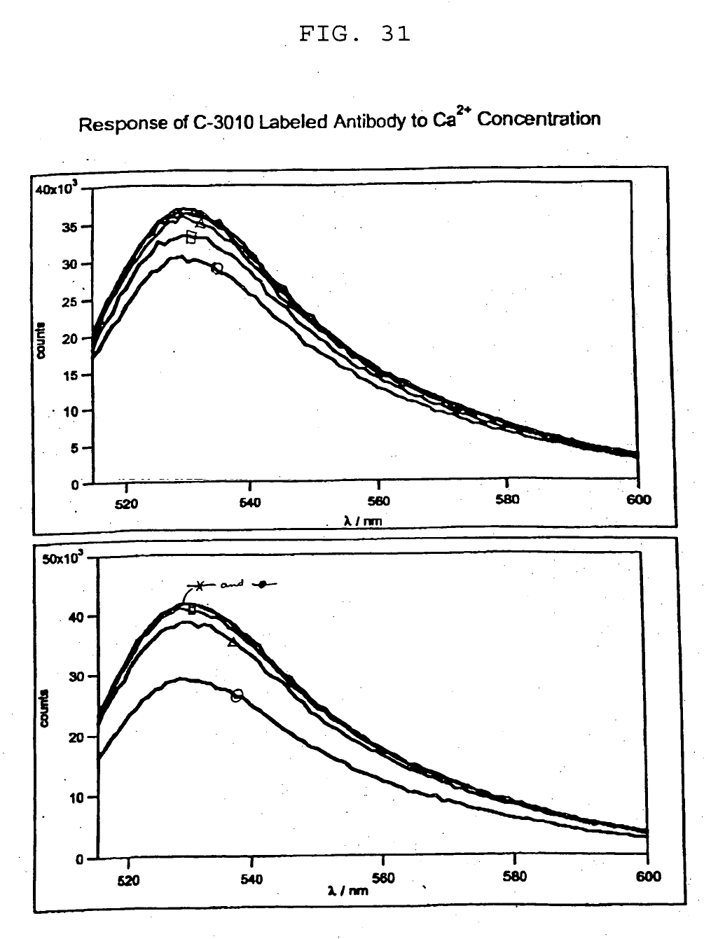

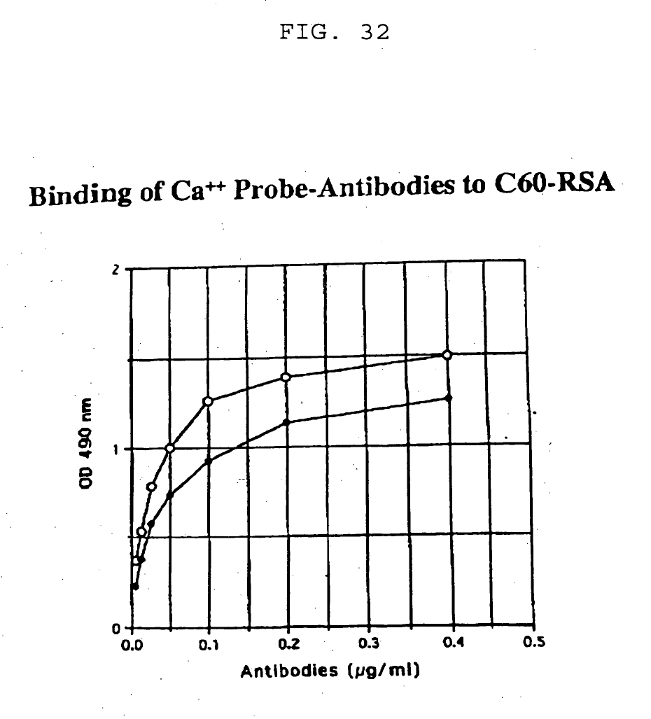

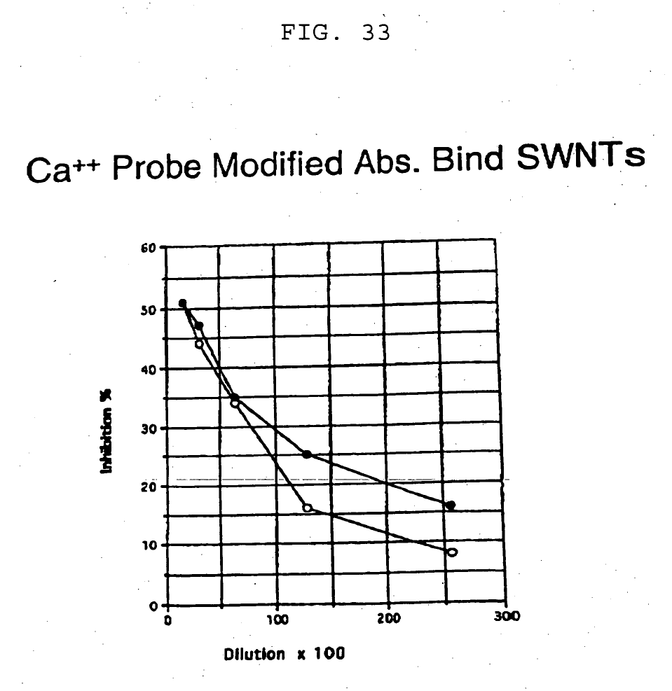

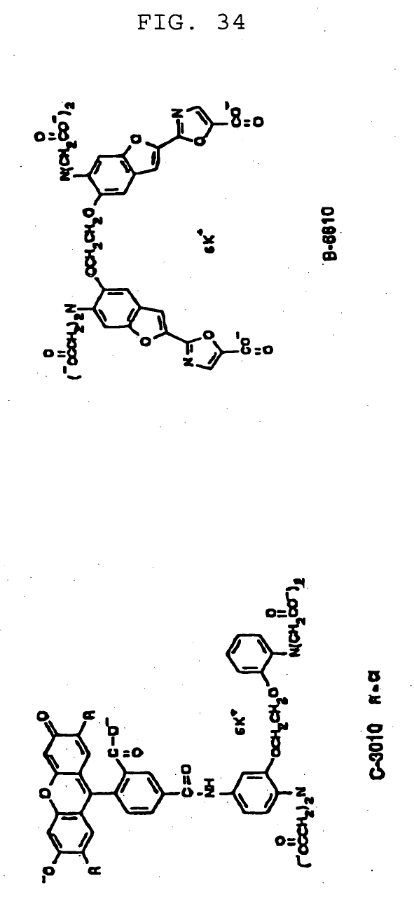

C₆₀ Antibodies on SWNTs were directly imaged by atomic force microscopy. SWNT ropes on mica were initially imaged, the surface was then exposed to antibody solution, and finally, the same SWNT was imaged again in air. This sequence distinguishes any preexisting surface particles from bound antibodies. Three drops of aqueous SWNT suspension (0.064 mg/mL in Triton 100× surfactant, from Tubes@Rice) were spun onto a freshly cleaved mica surface. The sample was imaged by tapping mode AFM in air, as shown in FIG. 24a. One drop of fullerene-specific antibody (0.001.25 mg/mL in 10 mM phosphate buffer, 150 mM NaCl, pH 7.3) was then deposited onto the surface. Liquid was removed by glass piper after 8 min, and the sample was dried in air. The sample was then washed with 60 drops of water while spinning to remove weakly bound, physisorbed antibody layers. The same SWNT was then imaged in FIG. 24. A significant number of objects are adsorbed on the nanotube, in previously clean regions, and on the mica substrate. A higher resolution perspective surface plot in FIG. 24c shows antibodies on the nanotube and on mica. The mica antibody images are similar in height, width, and dimpled appearance to those previously reported for monoclonal mouse IgG1 on mica (10). A before and after control experiment with a non-fullerene-specific monoclonal antibody, H413 (specific for aldosterone receptor (11)), yielded antibody aggregates on the mica but left the nanotube clean.

In a separate experiment, we mixed an aqueous SWNT suspension with an anti-fullerene antibody solution and then imaged the SWNTs on mica. One drop of antibody solution (0.25 mg/mL) was mixed with 5 mL of SWNT suspension (0.064 mg/ml) and cooled at 5° C. for 2 h. Three drops of this mixture were spun on a freshly cleaved HOPG surface (Highly Ordered Pyrolytic Graphite) and imaged without washing. FIG. 24d shows many SWNT-adsorbed antibody images similar to those in FIG. 24c.

We conclude that this monoclonal IgG C₆₀-specific antibody specifically binds to aqueous carbon SWNT ropes. SWNTs have a curved, hydrophobic, n-electron-rich graphitic surface² analogues to that of C₆₀ itself; the hydrophobic binding site of the antibody is sufficiently flexible to recognize both. Our work bridges two disparate disciplines: electrical nanotechnology and monoclonal immunology (12). A combination of the extensively developed methods of both fields can have practical consequences. For example, the antibody-coated SWNTs can be used as probes of cell or membrane function. An SWNT rope has a diameter of roughly 10 nm, far smaller than present metallic or glass capillary intracellular probes. They should be capable of insertion into and withdrawal from specific regions of some cells, hopefully with minimal disturbance of cell or membrane function. The anti-fullerene antibody on the surfaces of carbon nanotubes can be covalently decorated with probes of cell function, e.g., redox or luminescent probes (e.g., for Ca²⁺) After insertion, the probe molecule(s) can be optically excited or electrically addressed via the conducting SWNT wire. Unlike most semiconductors and metals, SWNTs do not form insulating surface oxides at room temperature. There is direct electrical contact with the antibody, as would occur at a Au electrode; indeed, recent experiments have demonstrated that nanotube electrical properties change with reversible adsorption of molecular species (13, 14).
1. Ujima S., Ichihoshi T. Nature (1993) 363: 603-605.
2. For a review see: Saito, R., Drosselhaus, G., Drosselhaus, M. S. Physical Properties of Carbon Nanotubes, Imperial College Press: Singapore, (1998).
3. For a review see: Woolley, A., et al. Chem Biol. (2000) 7: R193-R204.
4. Ches, R. J., Zhang, Y J., Wang, D., Dui, IL, J. AM. Chem. Soc. (2001) 123: 3838-3839.
5. Guo, Z. Sadler, P. J., Tsang, S. C., Adv. Mater. (1998) 10: 701-703.
6. Balvoine F., et al. Angew. Chem, Int. Ed. Engl. (1999) 38: 1912
7. Chen, B.-X., Wilson, S. R., Das, M., Coughlin, D. J., Erlanger, B. F. Proc. Natl. Acad. Sci. U.S.A. (1998) 95: 10800-10813.
8. Braden, B. C., et al. Proc. Natl. Acad. Sci. U.S.A. (2000) 97: 12193-12197.
9. Engvall, E. Methods Enzymol. (1990) 70: 419-439.
10. Bergk, M., et al. J. Colloid Interface Sci. (1998) 206: 475-481.
11. Lombas, M., Edelman, J. S., Erlanger, B. F., J. Biol.

Chem. (1989) 264: 2528-2536.
12. For a review see: Lang, Van Vunakis, H., Monoclonal Antibodies and. General Immunoassay Methods, Immunochemical Techniques, Colowich, S. P., Kapla Immunochemical Techniques N. O., Eds., Methods in Enzymology 92, Part E: Academic Press: New York, (1983).
13. Kong, J., et al. Science (2000) 287: 622-625.
14. Yu, Z., Brus, L. E., J. Phys. Chem. (2000) A104: 10995-10999.
   Sixth Series of Experiments
   Fullerenes and Nanotubes

Until 1985 there were only two known allotropic forms of carbon: graphite and diamond. In 1985, a novel allotrope was reported in which 60 carbon atoms were arranged as a truncated icosahedron, with 60 vertices and 32 faces, 12 of which were pentagonal and 20 hexagonal (1). It was dubbed Buckminsterfullerene (usually shortened to fullerene) because of its geodesic character, a name that has held through the present day.

Considerable activity followed this discovery, particularly after procedures were developed to prepare fullerenes in workable quantities (2, 3). Various fullerene-based compounds have been prepared, and diverse uses were sought for them. Some were incorporated into photovoltaic cells (4) and nanotubes (5). Others were tested for biological activity (6), including antiviral (7, 8), antioxidant (9, 10), and chemotactic activities (11), and as neuroprotective agents in a mouse model of amyotrophic lateral sclerosis (12).

The discovery of a new form of carbon fullerene—fullerene-C60—in 1985 by Smalley and coworkers at Rice University stimulated tremendous world-wide research interest (35). When the Huffman-Kratschner (HK) arc process for production of fullerene-C60 in quantity made samples of C60 available in 1991, scientists throughout the world began to explore C60 chemistry.

Close relatives of C60, nanotubes (very long graphitic molecular fibers), were first observed in the HK arc process by Iijima (36). Numerous theoretical and microscopic studies of nanotubes followed this exciting discovery (37). However, it was the recent breakthrough by Smalley—preparation of gram quantities of nanotubes—that promised to propel nanotube research to commercial application. Nanotubes are called “the perfect carbon fibers.” They conduct electricity as well as qold, and are 100 times stronger than steel at ⅙ the density. Important DoD-related applications include mechanical/chemical applications as well as optical/electronic applications. Possible mechanical/chemical uses are light weight, high strength composites for vehicle body panels, ship hulls and airframes, ship and helicopter propellers, rocket nozzles, helmets and body armor. Possible electronic/optical applications include tactical displays, laser eye protection, vacuum electronics, capacitors, batteries and fuel cells. Other uses include chemical filters, catalyst supports, hydrogen storage and nanoscale devices for computation (38).

The enormous promise of nanotubes requires the development of many supporting basic science areas to assist in characterization and to begin evaluation of potential biological applications and potential health risks. Considerable work is known about C60 chemistry and much work has been done on toxicology and even drug development with fullerenes (39). The toxicology of large carbon fibers has been extensively studied and a review is available (40). Nothing at all is known about the toxicology of nanotubes or their interactions with biological systems. While no general toxic effects of fullerenes are known, the small diameter and very large aspect ratio (ratio of length to diameter) of nanotubes are somewhat reminiscent of asbestos fibers, and make an important side-benefit of this research the beginning steps to understanding the relationship of nanotubes to human health.

Antibodies

The development of methods for the preparation of antibodies to hydrophobic small molecules is well known to one of skill in the art (41). During the 1950's and 60's, Erlanger developed the approach of linking steroid molecules to serum albumins to immunize rabbits with the steroid-serum albumin conjugates and thereby obtain specific anti-steroid antibodies. This method has revolutionized detection of hydrophobic compounds in biological systems. Erlanger has also prepared MABs to the hydrophobic molecule taxol, as well as a MAB that mimics taxol (42).

The clonal selection theory tells us that antigens elicit the production of antibodies by selecting for specific antibody producing cells already present in the repertoire of immunized animals (13). Although there is debate about the size of the “available” repertoire (14, 15), immunologists usually work on the assumption that the repertoire is diverse enough to be counted on,” to produce antibodies to “any” molecule a researcher may choose. This is, of course, an unreliable assumptions as experimental failures rarely find their way into the literature. The question that arose, therefore, is whether the immune repertoire is “complete” enough (15) to recognize and respond to the unprecedented geodesic structure of the fullerenes or sufficient aspects of it—more particularly, whether the immune system can process a fullerene-protein conjugate and display the processed peptides for recognition by T cells to yield antibodies. It has been shown that it does (34) and produced a mouse monoclonal anti-fullerene antibody hybridoma designated 1-10F-8A (ATCC Accession No. PTA-279) which produces a monoclonal antibody that binds to fullerenes.

Applications of Antibodies and Napotubes

Anit-nanotube antibodies have use in the assembly and orientation of nanotubes into useful nanoscale devices on the surface of silicon chips (45). Because nanotubes can be manipulated, e.g. by an atomic force microscope (46), and because of their extremely small diameter (in the order of nanometers: 10⁻⁹ meters), they can also serve as ideal probes of intracellular activity, i.e. they can be introduced into cells with minimal disturbance of cellular activity. Probes now in use are mainly glass capillary tubes drawn to a fine tip, the diameters of which are orders of magnitude greater than that of a carbon nanotube.

Experimental Details

I. Materials, Methods and Results

A. Method of Making Anti-Fullerene Antibodies and Anti-Nanotube

Antibodies

Antibodies that bind to fullerenes and nanotubes and the method of making same are described supra.

B. The Anti-Fullerene Antibody Binds Nanotubes

The following is a description of the ELISA immunoassay showing binding of a nanotube preparation to anti-fullerene antibody (i.e., an antibody known to bind fullerenes) by inhibition of binding of the fullerene compound to monoclonal anti-fullerene antibody.

The anti-fullerene antibody produced by the hybridoma 1-10F-8A (ATCC Accession No. PTA-279) (0.5 micrograms in 0.1 N sodium bicarbonate solution) was used to coat 96 well round bottom plastic plates (Corning #25855).

A solution of biotinylated C₆₋₀-rabbit serum albumin (—RSA) conjugate (0.7 mg/ml) with about 12 C60 groups per molecule was diluted 10,000 times in phosphate-buffered saline (PBS) containing 0.05% Triton X-100.

Five microliters of a single wall nanotube suspension (3.4 mg/ml) in Triton X-100 was diluted 100-fold in distilled water. After centrifuging to remove the larger particles in suspension, the supernatant was diluted 160 times in PBS-0.5% Triton-X100. Various dilutions of this colloidal-like suspension were mixed with equal amounts of the biotinylated C-60 RSA described in step 2.

Equal volumes (100 microliters) of the various solutions made up in step 3 were added to the antibody-coated wells. After 3 hours during which the nanotubes were competing with the biotinylated C60-RSA for binding to the anti-fullerene antibody, the plates were washed with PBS and the amount of biotinylated C60-RSA bound to the immobilized antibody was determined by the ability to bind avidin-peroxidase by a colorimetric assay using o-phenylenediamine as the peroxidase substrate.

The principle of the above assay is to measure the ability of nanotubes to compete with a fullerene derivative for binding to the anti-fullerene antibody. FIG. 29 quantifies the competition and proves that nanotubes compete very successfully with a fullerene compound for binding to an anti-fullerene antibody.

C. Conjugation of Calcium Probes to Monoclonal Anti-Fullerene Antibody

Fluorescent calcium ion probes, B-6810 and C-3010 (Molecular Probes, Inc., Eugene, Oreg.) (structures are shown in FIG. 34), were conjugated to the monoclonal antibody produced by the hybridoma l-10F-8A (ATCC Accession No. PTA-279). C-3010 (400 μg) was dissolved in 50 μl of distilled water. Five microliters of N HCl was added to convert C-3010 to its acid form, which precipitated from solution. The supernatant was removed by centrifugation, the precipitate washed with distilled water and dried overnight in a desiccator over concentrated sulfuric acid and pellets of sodium hydroxide.

The dried, acid form of C-3010 was dissolved in 200 μl of dry pyridine. Then, 360 μg of dicyclohexycarboiimide in 100 μl of dry pyridine was added. The reaction mixture was allowed to stand for 48 hours at room temperature. It was then added dropwise, with stirring to 2.5 mg of the monoclonal antibody produced by the hybridoma 1-10F-8A (ATCC Accession No. PTA-279) in 0.1 N NaHCO3 (adjusted to pH 9.4). The reaction at room temperature was allowed to proceed for 3 hours, after which the reaction mixture was dialyzed against phosphate buffered saline, pH 7.5, for 3 hours.

The final product, as determined spectrophotometrically, was a monoclonal antibody containing ca. 5 moles of C-3010 per mole of antibody.

The covalent linkage of B-6810 to anti-fullerene was carried out similarly. As with C-3010, there were 5 moles of B-6810 per mole of antibody.

II. Discussion

In these experiments, two Ca⁺⁺ probes, B-6810 and C-3010, were covalently attached to anti-fullerene antibodies, i.e., one of the probes to each antibody. It was determined that there were about 5 probe molecules per antibody and that the antibodies still could bind to fullerenes and carbon nanotubes. Each probe, upon stimulation by light of a particular wavelength, fluoresces in the presence of Ca⁺⁺, the fluorescence being related to the concentration of Ca⁺⁺. It was shown that they do the same when covalently attached to anti-fullerene antibodies. (See FIGS. 30-31). In other words, the reagent covalently bound to the antibody behaves as if it were the free Ca⁺⁺ probe.

In addition, it was shown that the Ca⁺² probe-labeled antibodies are still capable of binding to fullerenes (see FIG. 32) and nanotubes (see FIG. 33).

### REFERENCES FOR SIXTH SERIES OF EXPERIMENTS

1. Kroto, H. W., Heath, J. R., O'Brien, S. C., Curt, R.

F. & Smalley, R. E. (1985) Nature (London) 318, 162-163.
2. Kratschmer, W., Lamb, L. D., Fostiropoulos. K. & Huffman, D. R. (1990) Nature (London) 347, 354-357.
3. Scrivens, W. A., Bedworth. P. V. & Tour, J. M. (1992) J. Am. Chem. Soc. 114, 7917-7919.
4. Yu. G., Gao, J., Hummelin,. J. C., Wudl, F. & Heeger, A. J. (1995) Science 270, 1789-1791.
5. Yacobson, B. I. & Smalley, R. E. (1997) Amer. Sci. 85, 324-337.
6. Jensen, A. W., Wilson, S. R. & Schuster, D. I. (1996) Biorg. Med. Chem. 4, 767-779.
7. Sijbesma, R., Srdanov, G. Wudl. F., Castoro, J. A., Wilkins, C., Friedman, S. H., DeCamp, D. L. & Kenyon, G. L. (1993) J. M. Chem. Soc. 115, 6510-6512.
8. Schinazi, R. F., Sijbesma. R., Srdanov, G., Hill, C. L. & Wudl, F. (1993) Antimicrob. Agents Chemother. 37, 1707-1710.
9. Chiang, L. Y., Lu, J. T. & Lin, J. T. (1995) J. Chem. Soc. Chem. Commun., 1283-1284.
10. Lu, L.-H., Lee, Y.-T., Chen, H.-W. Chiang, L. Y. & Huang, H.-C. (1998) Brit. J. Pharmacol. 123, 1097-1102.
11. Toniolo, C., Bianco, A., Maggini, M., Scorrano, G., Prato, M., Marastoni, M., Tomatis, R., Spisani, S., Palu, G. & Blair, E. D. (1994) J. Med. Chem. 37, 4558-4562.
12. Dugan, L. L., Turetsky, D. M., Du, C., Lobner, D., Wheeler, M., Almi, C. R., Shen. C. K.-F., Luh, T.-Y, Choi, D. & Lin, T.-S. (1997) Proc. Natl. Acad. Sci. USA 94, 9434-9439.
13. Jerne, N. K. (1974) Ann. Inst. Pasteur/ImmtLnol 125, 373-389.
14. Rajewsky, K., Forster, I. & Kumano, A. (1987) Science 238, 1088-1094.
15. Langman. R. E. (1992) Int. J. Clin. Lab. Res. 22, 63-68.
16. Wang, Y., Cao, J., Schuster, D. I. & Wilson, S. R. (1995) Tetrahedron Lett. 36, 6843-6846.
17. Bensasson. R. V., Bienveue, F., Janot, J.-M., Leach, S., Seta, P., Zhao, H., Schuster, D. I. & Wilson, S. R. (1995) Chem. Phys. Lett. 248,566-570.
18. Anderson, T., Nilsson, K., Sundahl, M., Westman, G. & Wennerstrom, O. J. (1992) J. Chem. Soc. Chem. Comm., 604-606.
19. Boulas, P., Kutner, W., Jones, M. T. & Kadish. K. M. (1994) J. Phys. Chem. 98, 1282-1287.
20. Snyder, S. L. & Sobocinski, P. Z. (1975) Anal. Biochem. 64, 284-288.
21. Erlanger, B. F. & Brand, E. (1951) J. Am. Chem. Soc. 73, 4026-4027.
22. Johnstone, A. & Thorpe, R. (1982) Immunochemistry in Practice (Blackwell Scientific, Oxford), pp. 122-125.
23. Ku, H. H., Cleveland, W. L & Erlanger, B. F. (1987) J. Immunol. 139, 2376-2384.
24. Janeway, C. A., Jr. & Travers, P. (1996) Immunobiology (Garland, N.Y.), pp. 8:3-8:5.
25. Erlanger, B. F., Borek, F., Beiser, S. M. & Lieberman, S. (1959) J. Biol. Chem. 234, 1090-1094.
26. Arevolo, J. H., Hassig, C. A., Stura, M. J., Taussig, M. J. & Wilson, I. A. (1994) J. Mol. Biol. 241, 663-690.
27. Haddon, R. C. (1993) Science 261, 1545-1550.
28. Meidine, M. F., Hitchcock, P. B., Kroto, H. W., Taylor. R. & Walton, D. R. M. (1992) J. Chem. Soc. Chem. Comm., 1534-1537.
29. David, W. I. F., Ibberson, R. M., Mattewman, J. C., Prassides, K., Dennis, T. J. S., Hare, J. P., Kroto, H. W., Taylor, R. & Walton. D. R. M. (1991) Nature (London) 353, 147-149.
30. Hirsch, A., Lamparth, I. & Grosser, T. (1994) J. Am. Chem. Soc. 116,9385-9386.
31. Skiebe, A., Hirsch, A., Klos. H. & Gotschy. B. (1994) Chem. Phys. Lett. 220, 138-140.
32. Subramanian, R., Boulas, P., Vijayshee, M. N., D'Souzaj. F. & Jones, M. D. (1994) J. Chem. Soc. Chem. Commun., 1847-1848.
33. Friedman, S. H., DeCamp, D. L. Sijbesma, R. P., Srdanov. G., Wudl, F. & Kenyon, G. L. (1993) J. Am. Chem. Soc. 115, 6506-6509.
34. PCT International Application No. PCT/US00/23629, filed Aug. 29, 2000, International Publication No. WO 01/16155, published Mar. 8, 2001 on behalf of The Trustees of Columbia University In The City Of New York.
35. Billips, W. E., Clufolini, M. A., Eds, “Buckminsterfullerene,” VCH, NY (1992).
36. Iijima, E., Ichihashi, T., “Single-shell carbon nanotubes of 1-nm in diameter,” Nature 361: 603 (1993).
37. “Carbon Nanotubes,” Ann. Rev Material Sci., 243: 235 (1994).
38. Yokabsen, B. I., Smalley, R. E., “Fullerene Nanotubes: C_{1,000,000} and Beyond,” American Scientist, 85: 324 (1997).
39. Jenson, A., Wilson, S. R., Schuster, D. I., “Biological Applications of Fullerenes-A Review,” Bioorganic and Medicinal Chemistry, 4:767 (1996).
40. Thomson, S. A., “Toxicology of Carbon Fibers” Appl. Ind. Hyg. 29 (1989).
41. Erlanger, B. F., “Preparation of Antigenic Hapten-Carrier Conjugates,” Methods in Enzymology 70:85 (1980).
42. Leu, J-G, Chen, B-X, Diamanduras, A. W., Erlanger, B., “Idiotypic mimicry and the assembly of a supramolecular structure, and anti-idiotypic antibody that mimics taxol in its tubulin-microtubule interactions,” Proc. Natl. Acad. Sci. USA, 91:10690 (1994).
43. Lamb, L. D. et al., “Mass spectrometry and STM Imaging of Giant Fullerenes,” Science 255:1413 (1992).
44. Cytometry, 14:265 (1993).
45. Klibanov, A. M., “Enzymic Catalysis in Anhydrous Organic Solvents,” Trends Biochem Sci, 14: 145 (1989).
46. Wong et al., “Covalently functionalized nanotubes as nanometresized probes in chemistry and biology”, Nature 394: 52-55 (1998).
47. Neurosci. Lett. 207:17 (1996).
48. Guide to Electroporation and Electrofusion, D.C. Chang et al., eds., Academy Press (1992).
49. Meth. Cell Biol. 29:153 (1989).
50. U.S. Pat. No. 4,816,567, issued Mar. 28, 1989 to Cabilly et al.
51. U.S. Pat. No. 5,225,539, issued Jul. 6, 1993 to Gregory Winter.
52. U.S. Pat. No. 5,585,089, issued Dec. 17, 1996 to Queen et al.
53. U.S. Pat. No. 5,693,761, issued Dec. 2, 1997 to Queen et al.
54. PCT International Application No. PCT/US89/05857, filed Dec. 28, 1989, published Jul. 26, 1990, WO 90/07861.

1. A composition comprising a nanotube and at least one anti-nanotube antibody, wherein the anti-nanotube antibody is bound to the nanotube.

2. A composition comprising a fullerene and at least one anti-fullerene antibody, wherein the anti-fullerene antibody is bound to the fullerene.

3. The composition of claim 1 or 2, wherein the antibody is a monoclonal antibody or an antigen-binding portion thereof.

4. The composition of claim 3, wherein the monoclonal antibody or antigen-binding portion thereof binds to the same epitope on the nanotube as the monoclonal antibody produced by the hybridoma designated 1-10F-8A (ATCC Number PTA-279).

5. The composition of claim 3, wherein the monoclonal antibody or antigen-binding portion thereof competitively inhibits the binding to the nanotube of the monoclonal antibody produced by the hybridoma designated 1-10F-8A (ATCC Number PTA-279).

6. The composition of claim 4 or 5, wherein the monoclonal antibody or antigen-binding portion thereof is the monoclonal antibody produced by the hybridoma designated 1-10F-8A (ATCC Number PTA-279) or the antigen-binding portion thereof.

7. The composition of claim 1 or 2, wherein the antibody is a polyclonal antibody.

8. The composition of claim 1 or 2, wherein the composition is immobilized.

9. The composition of claim 1, wherein the nanotube has bound thereto a plurality of anti-nanotube antibodies.

10. The composition of claim 2, wherein the fullerene has bound thereto a plurality of anti-fullerene antibodies.

11. The composition of claim 1 or 2 further comprising a moiety, wherein the moiety is bound to the antibody.

12. The composition of claim 11, wherein the moiety is selected from the group consisting of a detectable marker, a probe, a small molecule, a polypeptide, an antibody and a nucleic acid.

13. The composition of claim 12, wherein the detectable marker is selected from the group consisting of a radioactive label, and a calorimetric., luminescent, or fluorescent marker.

14. The composition of claim 12, wherein the probe permits the detection of ion concentration.

15. The composition of claim 14, wherein the ion is Ca⁺².

16. The composition of claim 14, wherein the probe is C-3010 or B-8610.

17. A method for introducing the composition of claim 1 or 2 into a cell comprising contacting the composition with the cell under conditions permitting entry of the composition into the cell.

18. The method of claim 17, wherein the conditions permitting entry of the composition into the cell comprises the use of an atomic force microscope.

19. A method for determining whether an agent is present in a sample comprising contacting the sample with the composition of claim 11, wherein the moiety of the composition permits the detection of the agent, and detecting any agent present in the sample via the moiety, thereby detecting whether the agent is present in the sample.

20. A method for introducing a moiety into a sample comprising introducing into the sample the composition of claim 11, wherein the moiety being introduced into the sample is the moiety of the composition.

21. A kit comprising the composition of claim 1 or 2 and instructions for use.

22. A kit comprising the composition of claim 11 and instructions for use.

23. A kit comprising a nanotube, an anti-nanotube antibody, and instructions for making and/or using the composition of claim 1.

24. A kit comprising a fullerene, an anti-fullerene antibody, and instructions for making and/or using the composition of claim 2.

25. A kit comprising the composition of claim 1 or 2, a moiety, and instructions for binding the moiety to the antibody of the composition.

26. A method for immobilizing a nanotube on a solid support comprising contacting the composition of claim 1 with a solid support having affixed thereto an agent that binds to the antibody of the composition, under conditions permitting such binding, thereby immobilizing the nanotube.

27. A method for immobilizing a nanotube on a solid support comprising contacting the composition of claim 11 with a solid support havinq affixed thereto an agent that binds to the moiety of the composition, under conditions permitting such binding, thereby immobilizing the nanotube.

28. A method for immobilizing a fullerene on a solid support comprising contacting the composition of claim 2 with a solid support having affixed thereto an agent that binds to the antibody of the composition, under conditions permitting such binding, thereby immobilizing the fullerene.

29. A method for immobilizing a fullerene on a solid support comprising contacting the composition of claim 11 with a solid support having affixed thereto an agent that binds to the moiety of the composition, under conditions permitting such binding, thereby immobilizing the fullerene.

30. The method of claim 26, 27, 28, or 29, wherein the solid support has the agent affixed thereto at one or more discrete loci.


SEQUENCE LISTING (4 sequences)

SEQ ID NO: 1 - DNA, 404 bp, Mouse
atgggatgcagctggggcatgctcttcctcctgtcaataactgcaggtgtccattgccaggtccacctacaacaatctggacctgagctggtgaggcctggggcctcagtgaagatttcctgcaaaacttctggctacgtattcagtagttcttggatgaactgggtgaaacagaggcctggacagggtcttaagtggattggacgaatttatcctggaaatggaaatggaaatactaattacaatgagaaattcaagggcaaggccacactgactgcagacaaatcctccaacacagcctacatgcagctcagcagcctgacctctgtggactctgcggtctatttctgtgcaacatcctcggcttactggggccaagggactctgctcactgtctctgcagc

SEQ ID NO: 2 - protein, 132 aa, Mouse [murine]
MGCSWGMLFLLSITAGVHCQVHLQQSGPELVRPGASVKISCKTSGYVFSSSWMNWVKQRPGQGLKWIGRIYPGNGNTNYNEKFKGKATLTADKSSNTAYMQLSSLTSVDSAVYFCATSSAYWGQGTLLTVSA

SEQ ID NO: 3 - DNA, 324 bp, Mouse
gatatccagatgacacagactacatcctccctgtctgcctctctgggagacagagtcaccttcagttgcagtgcaagtcaggatattaacaattatttaaactggtatcagcagaaaccagatggaactattaaactcctaatctattacacatcaagtttacgctcaggagtcccatcaaggttcagtggtagtgggtctgggacagattattctctcaccatcaacaacctggaacctgaagatattgccacttatttttgtcagtatagtaggcttccgttcacgttcggctcggggacaaagttggaaataaaacgtaag

SEQ ID NO: 4 - protein, 109 aa, Mouse
DIQMTQTTSSLSASLGDRVTFSCSASQDINNYLNWYQQKPDGTIKLLIYYTSSLRSGVPSRFSGSGSGTDYSLTINNLEPEDIATYFCQQYSRLPFTFGSGTKLEIKRK